(19) United States
(12) Patent Application Publication
(10) Pub. No.: US 2023/0223126 A1
(43) Pub. Date: Jul. 13, 2023

(54) Digital Health Platform with Prescription Management and Integrated E-Commerce Curation

(71) Applicant: Azova, Inc., Alpine, UT (US)

(72) Inventor: Cheryl Lee Eberting, Alpine, UT (US)

(21) Appl. No.: 18/046,126

(22) Filed: Oct. 12, 2022

Related U.S. Application Data
(63) Continuation-in-part of application No. 16/244,883, filed on Jan. 10, 2019, now abandoned.
(60) Provisional application No. 62/722,926, filed on Aug. 26, 2018.
(60) Provisional application No. 62/615,954, filed on Jan. 10, 2018.
(60) Provisional application No. 62/765,480, filed on Aug. 25, 2018.

(51) Int. Cl. G16H 20/10; G16H 80/00; G06Q 30/0601

(52) U.S. Cl. CPC G16H 20/10; G16H 80/00; G06Q 30/0641

(57) ABSTRACT

A digital health platform enables the creation of a curated selection of products available for recommendation by healthcare providers and/or purchase by patients. The digital health platform may further facilitate electronic medical record management, physician-customizable online portals, patient services, health-related online marketplaces, secure messaging services, vaccine management, on-demand translations, real-time sales support, and the like. The selection of a product for recommendation or purchase may be linked to the scheduling of a telemedicine consultation related to the selected product. A healthcare practitioner may provide immediate click-to-buy links to various products and services to facilitate an improved user-experience and increase compensation available to healthcare providers.

### RELATED APPLICATIONS

This Application is a Continuation-in-Part of U.S. patent application Ser. No. 16/244,883, filed Jan. 10, 2019, titled “Digital Health Platform For Chronic Disease Management, Secure Messaging, Prescription Management, And Integrated E-Commerce Curation,” which claims priority to U.S. Provisional Patent Application No. 62/615,954, filed Jan. 10, 2018, titled “Secure Prescription Messaging,” U.S. Provisional Patent Application No. 62,765,480, filed Aug. 25, 2018, titled “Secure Prescription and Commerce Messaging,” and U.S. Provisional Patent Application No. 62/722,926, filed Aug. 26, 2018, titled “Secure Prescription and Commerce Messaging,” each of which is hereby incorporated by reference in its entirety, including any appendices associated therewith. This application also incorporates U.S. patent application Ser. No. 15/597,102, filed on May 16, 2017, titled “Telemedicine Platform with Integrated E-Commerce and Third Party Interfaces” in its entirety, to the extent that it is not inconsistent herewith.

### TECHNICAL FIELD

This disclosure relates to digital health platforms, such as telemedicine platforms, that incorporate various services and subsystems, including chronic disease management subsystems, electronic medical record management, physician-customizable online portals, patient services, health-related online marketplaces, and secure messaging services.

### BRIEF DESCRIPTION OF THE DRAWINGS

Non-limiting and non-exhaustive embodiments of the disclosure are described herein, including various embodiments of the disclosure illustrated in the figures listed below.

FIG. 1 illustrates a screenshot of one possible embodiment of a graphical user interface (GUI) of a healthcare provider selected product from a preselected product platform.

FIG. 2 illustrates a screenshot of one possible embodiment of a GUI for adding one or more products to a products platform.

FIG. 3 illustrates a screenshot of one possible embodiment of a GUI of a subset of products module utilized to display the subset of products from the curated products platform.

FIG. 4 illustrates a screenshot of one possible embodiment of a GUI of the healthcare provider utilizing the curated products platform to allow customizing a product.

FIG. 5 illustrates a screenshot of one possible embodiment of a GUI allowing a preview of an order request.

FIG. 6 illustrates one possible embodiment of a formulary builder tool interface.

FIG. 7 illustrates one possible embodiment of an extension of the formulary builder to allow for the addition of a compound.

FIG. 8 illustrates a screenshot of one possible embodiment of a GUI utilized to allow communication via a discussion module.

FIG. 9 illustrates a screenshot of one possible embodiment of a GUI utilized to allow communication via the discussion module.

FIG. 10 illustrates a screenshot of one possible embodiment of a GUI utilized to allow communication via the discussion module.

FIG. 11 illustrates a flowchart of a method for displaying preselected products consistent with embodiments of the present disclosure.

### DETAILED DESCRIPTION

A digital health platform may include any number of features and options that may be commonly used by all healthcare practitioners and/or patients and other features and options that are not used by certain subsets of healthcare practitioners and/or patients, whether due to personal preference, usability, cost, or inapplicability. Accordingly, the present systems and methods provide various aspects, features, services, and functions relating to telemedicine healthcare platforms and auxiliary services and products. It is appreciated that any of the various embodiments described herein may be combined in any number of ways and that all permutations and combinations of the described features, advantages, embodiments, and options are part of this disclosure, even if such permutations and combinations are not explicitly described in a single embodiment.

Disclosed herein are systems and methods for displaying preselected products that may be utilized in a variety of applications to allow healthcare providers access to these preselected products. In certain embodiments, a telemedicine system may be utilized to allow a first healthcare provider to create a curated products platform that displays products to be selected by a second healthcare provider. In some embodiments, the telemedicine system may be utilized to allow the second healthcare provider to select products from the curated products platform for a subset of products. In some embodiments, the subset of products may be displayed to and/or purchased by a third healthcare provider. The healthcare provider may include a physician, a pharmacy, a patient, or another user.

In various embodiments of the presently described systems and methods, the healthcare provider may create a customized, curated products platform by selecting one or more products to be displayed. Accordingly, rather than each healthcare provider being forced to use a generic platform or develop a fully customized platform from scratch, the healthcare provider may select from a list of available products (i.e., a product library) to create a customized platform, which can be referred to as the curated products platform. In some embodiments, products may include features of a digital health platform, functions of the digital health platform, interfaces of the digital health platform, prescriptions, medicines, or other products.

The present disclosure includes various embodiments utilizing the curated products platform to display a plurality of products from the product library based on the healthcare provider. Because an inability to review products based on the healthcare provider recommendation challenges the ability to review and/or purchase products, the curated products platform is able to display products specific to each healthcare provider. For instance, one preselected platform may display only skincare products, and another preselected platform may display only products related to broken bones. Each displayed product may pertain to one or more patients. Without the ability to display different products based on the healthcare provider on the curated products platform to each healthcare provider, the healthcare provider may not be able to find the correct product or may have to view numerous incorrect products before reaching the correct product. Additionally, selecting products from the curated products platform or from the subset of products saves time and improves reliability.

In various embodiments, third-party developers may develop applications for inclusion and/or incorporation into the telemedicine system. Third-party applications may pay to be listed on the telemedicine system and/or the telemedicine system may collect a percentage or other fee based on the usage and/or inclusion of each third-party application on a provider's customized, curated products platform.

Examples of third-party applications that can be integrated into the telemedicine system and/or selected for inclusion in a customized curated products platform by a healthcare provider include, but are not limited to: interfaces and monitoring software associated with biometric monitoring and/or tracking devices, patient engagement solutions, interfaces, programs, etc., electronic health record interfaces and portals, cardiology recovery and monitoring applications, laboratory interfaces, cholesterol management services, portals, interfaces and the like, lipid management and monitoring software solutions, asthma monitoring and management systems, blood pressure monitoring and management services and interfaces, weight loss management, support, monitoring, advisory systems and/or other third-party provider-patient interface, and monitoring and/or tracking solutions.

As a specific example, the healthcare provider may select (e.g., via an a La Carte or package subscription or one-time purchase) an application for integration or inclusion in the healthcare provider's curated products platform that provides a specific patient engagement platform. Based on the healthcare provider's specific needs, the integration and/or inclusion of specific products and services into the customized curated products platform may provide significant advantages to the medical providers, managers, and/or patients. For instance, a customized patient engagement application may be integrated into the telemedicine system to facilitate, control and/or manage how information is disseminated (e.g., via patient portals, hard copies, electronic communication, etc.), interactions between providers and patients (video, voice, messaging, store and forward, in-person, etc.), how data is collected or reported via testing and/or monitoring devices, and/or affect other patient engagement details.

As another example, a family physician may create a curated products platform and select a package of applications (e.g., a free package via a one-time purchase or as part of a subscription plan) that includes various patient interfaces and/or monitoring services associated with diet plans, cholesterol management, physical therapy, medication management solutions, and/or the like. In such embodiments, the family physician may utilize the telemedicine system to provide an online digital health platform that is more robust and offers services and products that the family physician might not otherwise be able to offer.

The curated products platform may be referred to and/or include a “dashboard” of features and/or services that are available to the healthcare provider including patients of an associated healthcare provider, family of patients of an associated healthcare provider, friends of patients of an associated healthcare provider, other associated or relevant healthcare providers from other healthcare facilities, and/or other entities. The dashboard of features and/or services available to each of these entities may be regulated and/or restricted based on the entity accessing the curated products platform, permission settings, and/or the assigned feature sets to each specific entity.

Any of a wide variety of health and wellness platforms may be integrated as products (potentially customizable) available for inclusion in a curated products platform. Such platforms include but are not limited to stress-reduction programs, weight loss counseling, group therapy sessions and the like that might be implemented (in person, via teleconference, via video conference, etc.) within or through integration with the telemedicine system.

As a specific example, a healthcare facility may create a customized, facility-specific, curated products platform that includes integration with a yoga instruction class taught online by a world-renowned specialist. Similarly, a practitioner-specific, curated products platform may provide a mechanism through which classes are taught or training is provided to groups of any size (patients or other physicians) regarding any of a wide variety of topics. Thus, healthcare providers may use the telemedicine system to create a customized conglomerate of existing services and platforms and couple them with any of the other products described herein. Some of the integrated services, platforms, products, and the like may be managed and provided by the service provider, while others may simply be integrated via screen-scrapes, links, APIs, and/or the like.

According to various embodiments, any of a wide variety of financial models may be used to charge for the use or inclusion of the services and features described anywhere herein. For example, fees may be paid by the healthcare provider to the service provider (i.e., the creator/supplier of the global product library), by a patient to the service provider, by the patient to a physician, and/or by the healthcare provider to the patient. The fees paid may be based on a subscription model, pay-per-use model, or based on a one-time fee. Any of a wide variety of tiered, discounted, incentive-based, and other financial models may be used. For example, in some embodiments, a variation of a concierge subscription model may be used where the patient pays the healthcare provider on a monthly or yearly basis for predetermined telemedicine and/or in-person services.

As an additional example, a service provider may charge a periodic (weekly, monthly, yearly, multi-year) fee to the healthcare provider based on the number and types of products selected for inclusion on the practitioner-specific, curated products platform. For instance, pricing models may be created for each product and/or for packages of product. In some embodiments, a flat pricing model may be implemented in which the healthcare provider pays the service provider a flat rate (one-time or subscription-based) to create the practitioner-specific, curated products platform with any number or type of product from the product library. In other embodiments, the pricing may be a la carte based on the specific products selected. In yet other embodiments, the pricing may be based on the actual usage of each of the selected product included in the practitioner-specific curated products platform.

In some embodiments, the healthcare provider may decide to charge patients directly for usage of the practitioner-specific curated products platform. In such an embodiment, a physician may generate a profit on the practitioner-specific curated products platform if the income received from the patients exceeds the fees charged by the service provider. As above, the physician may charge patients based on actual usage, the features/services used, under a subscription model, as a percentage of other healthcare costs, etc. Using any of the models described above, the healthcare provider may charge or bill an insurance company for the insurer's usage and/or the patient's usage of the practitioner-specific digital health platform. The ability to bill or the automatic billing of an insurance provider may be turned on or off on a visit-by-visit basis. In some embodiments, the healthcare provider may indicate that a visit is a follow-up telemedicine visit to an in-person visit. This may be important because some insurers and/or regulatory entities (e.g., a state) may require that first visits or periodic visits be conducted in person. The telemedicine system may dynamically adapt itself based on the zip code or other residency-identifying information and/or insurance information of the healthcare practitioner and/or patient to maintain compliance with all applicable laws and/or insurance rules.

In some embodiments, patients may create accounts with the service provider independent of the healthcare practitioners and/or insurance companies. Pricing models may vary based on the parties' interactions and/or affiliations with the service provider. For example, the fees charged to the healthcare practitioner and/or a patient may vary based on the pricing agreement of one or both parties with the service provider. For example, if the healthcare provider is a subscription-based customer of the service provider, any interaction by the patient with the healthcare practitioner may be free of charge. Whereas, if the healthcare provider is using a free, one-time payment, trial, discounted, split-fee arrangement, or another pricing model, an interaction by the patient with the healthcare practitioner may be billed or charged to the patient by the healthcare practitioner and/or directly by the service provider.

In addition to the products described above and regardless of the pricing model used, the product library may include any number of telemedicine features, functions, products, services, and/or the like that are known in the art of digital health platforms. These previously known telemedicine services, features, and functions may be recast as optionally selectable products that can be included in the product library and made available for selection by the healthcare provider for inclusion in a practitioner-specific curated products platform.

The curated products platform provides advantages to and may be used by any of a wide variety of people associated with healthcare, including, but not limited to: pharmacists, physicians (MDs), osteopathic physicians (DOs), nurse practitioners, physician's assistants, mental health professions, psychologists, social workers, mental health therapists, health and wellness professionals, dieticians, nutritionists, associated insurers, agents, billing specialists, patients, and/or other persons or entities associated with mental, beauty, aesthetic, physical, and other healthcare areas.

Many of the embodiments described herein reference a “healthcare provider” as an example of the entity that is creating, customizing, selecting, and/or otherwise utilizing the telemedicine system. However, it is appreciated that the entity actually managing, setting up, initializing, or otherwise involved with the telemedicine system may be a healthcare administrator, information technology (IT) specialist or other entity that does not necessarily treat, test, diagnose, or otherwise interface with patients. Such entities may be referred to as “providers” inasmuch as they provide services to patients, clients, and the like, and as “healthcare providers” inasmuch as they act on behalf of, under the direction of, for the benefit of those who actually treat, test, diagnose, or otherwise interface with patients.

In various embodiments, the telemedicine system may include and/or support custom, semi-custom, or standardized integration with one or more laboratories, imaging centers, and/or other healthcare-related facilities or service centers. The telemedicine system may include, or allow the healthcare providers to enable integration with any number of laboratories such as blood or pathology laboratories, or imagining centers such as radiology imaging centers and/or hospital imaging centers. For example, in some embodiments, the healthcare provider may customize a “provider dashboard” to include or otherwise indicate which of a plurality of laboratories and/or imaging centers are supported.

In various embodiments, the telemedicine system may include online open houses and/or monthly specials. For example, since the telemedicine system is used by providers and any other business, it may be beneficial to allow such entities to conduct online open houses and/or to promote and sell any specials

For example, an open house may allow the provider (or another customer of the telemedicine system) to offer remote consultations over the internet and to make a recommendation for appropriate services and specials to the prospective client online. The specials will be available to purchase in association with a consultation during the online open house promotion. When the provider configures an online open house, the provider may select and configure (choose the descriptions and price) for any visit type offered via the telemedicine system to be offered as part of the open house. The provider can also choose to charge a fee or to offer the consultation for free.

The specials that are offered as part of the open house may be completely customizable by the provider. As a specific example, the provider may have a tool on their dashboard that says “Promotions.” There may be a drop-down menu under this button that has “Customize Your On-Line Open House” and “Monthly Specials” where the provider can create specials that may include “buy one chemical peel and get one free,” or a particular product can be displayed at a particular discount. Any services of any professional could be configured to be on sale or promotional in this way. This curated products platform may also allow the provider to sell their own in-stock inventory from their “specials” website if desired.

Once the provider has configured their Online Open House and their monthly specials, the system may generate a widget (which will also be customized by the customer to match their website) that says “Our Monthly Specials” and “On-Line Open House Going on Right Now.” When a prospective client/patient clicks on the widget(s), a webpage may open displaying the monthly specials and the Online Open House products along with the interface to the telemedicine clinic. The top of the page for the open house and monthly specials may have “Get an Online Consultation. Find Out What Is Right for You!”

There may be a small image of an online consultation taking place on the right with a drop-down menu of the providers in the practice from which the patient can select the online consultation. The providers may be able to configure their consultation fees and the special prices of their consultation fees on their backend. The widget for the Online Open House can be placed anywhere on the provider's website including the home page and/or on a telemedicine clinic button.

The telemedicine system may include “Specials” and/or “Open House Discounts” pages or websites that conglomerate all the particular healthcare provider's curated products platform subscriber's ongoing specials into a single location. This may allow customers/patients to shop for discount products and services. In various embodiments, the telemedicine system may charge a referral fee for any leads/sales that are generated using this feature.

In various embodiments, the healthcare provider or organization may incorporate or link any of the various embodiments, functionalities, services, or the like via a button, link, or another graphical user interface (GUI) element on an existing website, application, program, or other user-accessible electronic content.

Various embodiments of the system and methods described herein allow prospective and existing customers or patients to browse products and services, some of which may be associated with an office consultation (in person or via telemedicine). Thus, in contrast to an independent e-commerce platform and a separate telemedicine consultation platform, the present systems and methods effectively provide or generate a composite website or platform that incorporates elements from a product/services sale page and telemedicine consultation offerings, allowing purchase of products/services directly recommended/selected by the healthcare provider, thus improving reliability and decreasing time.

The systems, methods, and platforms described herein may be adapted for use in other fields such as the legal, fitness, beauty, or another field. In such adaptations, the user may include an attorney, a yoga teacher, a hairstylist, or another user. In such adaptations, the products available for selection may be relevant to the field. Such products may further include a contract, a yoga session, a haircut, or other relevant product.

In one example, an inter-practitioner digital health platform includes multiple computing systems that interact with one another via a network. A first computing system may access a database of a pharmacist provider. The database includes and identifies a plurality of products available at a pharmacy. The pharmacy may, for example, include thousands of products that are categorized into hundreds of different categories. For example, the pharmacy may have hundreds of products that pertain to pain alleviation, hundreds of products that relate to skin care, hundreds of acne-related products, hundreds of foot-care products, hundreds of hygienic products, hundreds of allergy products, hundreds of bandages and wraps, hundreds of sleep aids, etc.

The system may display, via an electronic display, to the pharmacist provider, the plurality of available products via a first graphical user interface rendered by the first computing system. Again, the display may include thousands of products and they may be filterable, searchable, on different pages, and/or otherwise presented and available to the pharmacist. The system may also display information characterizing each of plurality of different healthcare practitioners, hospitals, doctors offices, etc. For example, the system may identify a general practice doctor at a first location, a dermatologist at a second location, and a pediatrist at a second location.

The system facilitates the unique curation of products. An input device associated with the first computing system may receive, from the pharmacist provider a first curated pharmacist-selection of a first subset of less than all the plurality of available products for selective presentation to the first healthcare practitioner, and a second curated pharmacist-selection of a second subset of less than all the plurality of available products for selective presentation to the second healthcare practitioner. The system may provide the first healthcare practitioner access to the first curated pharmacist-selection and to provide the second healthcare practitioner access to the second curated pharmacist-selection.

From the healthcare practitioner's perspective, they are presented with a relatively small pharmacy that specializes in products relevant to their specific practice. That is, the pediatrist sees a curation of products that are frequently prescribed by pediatrists. Simultaneously, the dermatologist sees dermatological products available for prescription or recommendation when a prescription is not required. A unified order requisition portal of the system (e.g., a module implemented as processor-executable instructions) receives from the first healthcare practitioner, a first recommended product from the first curated pharmacist-selection for purchase by a first patient and, receives from the second healthcare practitioner, a second recommended product from the second curated pharmacist-selection for purchase by a second patient.

The system renders for display on a first patient computing device, the first recommended product as recommended by the first healthcare practitioner alongside a plurality of additional products available at the pharmacy that are identified as being associated with the first recommended product and available for purchase by the first patient.

The system also renders for display on a second patient computing device, the second recommended product as recommended by the second healthcare practitioner alongside a plurality of additional products available at the pharmacy that are identified as being associated with the second recommended product and available for purchase by the second patient.

In another example, the system comprises a processor and a non-transitory computer readable medium with instructions stored thereon. When executed by the processor, the instructions cause a computing device to render a first graphical user interface, for display via an electronic display to a pharmacist provider. The first graphical user interface may display a plurality of products available for purchase from a pharmacy associated with the pharmacist provider. The first graphical user interface may also identify a first healthcare provider and a second healthcare provider, facilitate a curated selection of products by the pharmacist provider of a first subset of available products to be shared with the first healthcare provider, and facilitate a curated selection by the pharmacist provider of a second subset of available products to be shared with the second healthcare provider.

The system may integrate, via an integration subsystem, the first curated selection of available products into a first website of the first healthcare provider as an integrated graphical user interface, and the second curated selection of available products into a second website of the second healthcare provider as an integrated graphical user interface. The system may receive, via an order requisition portal, a first recommendation from the first healthcare provider to a first patient that the first patient purchase a first product in the first subset of available products integrated within the graphical user interface of the first website of the first healthcare provider. The portal may also receive a second recommendation from the second healthcare provider to a second patient that the second patient purchase a second product in the second subset of available products integrated within the graphical user interface of the second website of the second healthcare provider.

The system may receive requests from the first patient and the second patient to purchase the first product and the second product, respectively and, in response, generate orders within the pharmacy to fulfil the purchases of the first and second products by the first and second patients, respectively.

In another example, an inter-practitioner digital health platform includes a pharmacy database of a pharmacist provider that identifies thousands of products within hundreds of categories of products that are available from a pharmacy. A digital communications network connects the pharmacy database with remote computing systems. One such computing system may access the pharmacy database of the pharmacist provider and display, via an electronic display, to the pharmacist provider, the plurality of available products via a graphical user interface. The computing system may also display, via the electronic display, information identifying a first practice of a first healthcare practitioner and a second practice of a second healthcare practitioner.

An input device associated with the computing system may receive, from the pharmacist provider one or more curated pharmacist-selections of subsets of less than all the plurality of available products for selective presentation to different healthcare practitioners. An integration subsystem of the platform enables the pharmacist provider to share the curated pharmacist-selected subsets of available products with the different healthcare practitioners. An order requisition portal may receive, via the digital communications network recommendation of a products from the curated pharmacist-selected subsets of healthcare products for purchase by a patients directly from the pharmacy. Patients may confirm and complete the purchases of the recommended products which are then shipped to them. In some embodiments, a secure communication subsystem enables the pharmacist provider to communicate securely with the healthcare practitioners and the patients regarding the recommended products.

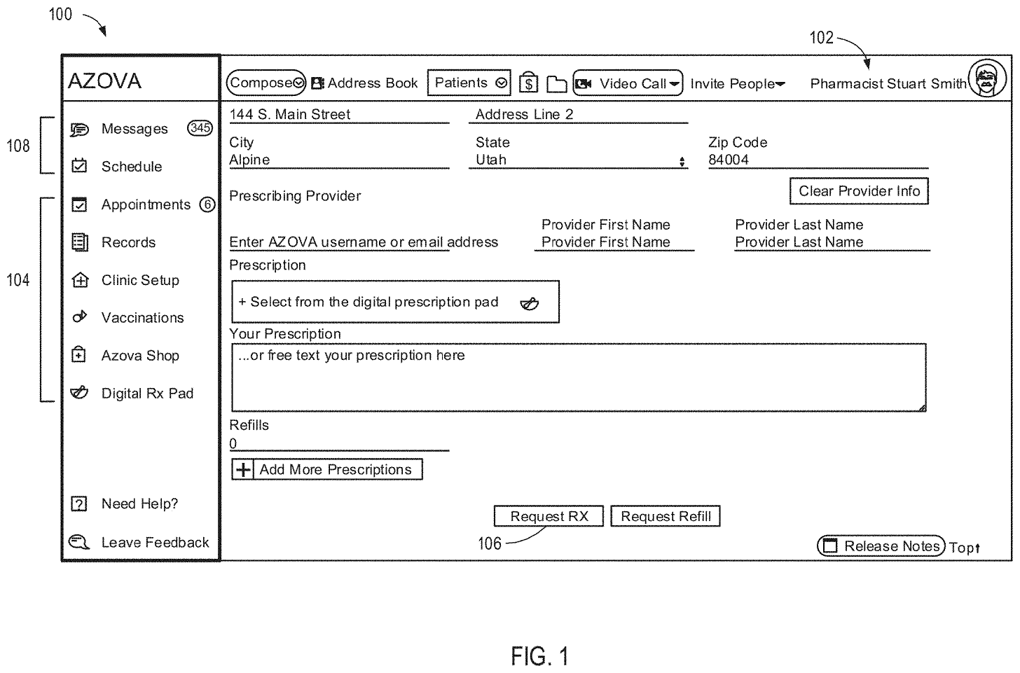

FIG. 1 illustrates a screenshot of one possible embodiment of a graphical user interface (GUI) of a healthcare provider 102 selecting a product 104 from a preselected product platform 100. The preselected platform 100 may include the one or more healthcare providers 102, the one or more products 104, an order requisition portal 106, and a discussion module 108. In some embodiments, the preselected platform 100 may be a physician's portal, a mini-store, healthcare facility inventory, or other platform associated with the healthcare provider 102. In some embodiments, the healthcare provider 102 may be a physician, a hospital, a healthcare facility, a pharmacy, a patient, or other entity/user associated with the products 104 on a display screen. In some embodiments, the curated products platform 100 displays the products 104. In some embodiments, the products 104 may include an appointment, a record, a product from a clinic, a vaccine, a product from a shop, a prescription, or other item. In some embodiments, the product 104 may include a traditional prescription, a digital prescription pad (Digital RxPad), and/or a prescription selected by utilizing a drop-down menu to allow selection of a category and a compound.

In some embodiments, the products 104 selected from a product library are displayed on the display screen for a first healthcare provider 102 to select from. This selected plurality of products is displayed for a second healthcare provider 102. The second healthcare provider 102 may select a subset of products from the plurality of displayed products. This subset of products is displayed for a third healthcare provider 102 or a patient. The third healthcare provider 102 or patient may select and/or purchase products 104 from the displayed subset of products. For instance, a pharmacy may set up a mini-store for a physician to select items. These items may then be sent to the physician's patient to ensure the patient is purchasing the correct items.

The subset of products may be received by the order requisition portal 106. The order requisition portal 106 may include fields for entering data associated with healthcare providers 102, products 104, and other related information. For instance, the order requisition portal 106 may collect data regarding the physician requesting the product, data regarding the requested product 104, and data regarding the patient for whom the product 104 is requested. In some embodiments, the orders requisition portal 106 receives the subset of products then displays the plurality of products to the healthcare provider 102. In some embodiments, the order requisition portal 106 determines if products 104 are available before displaying the products 104. For instance, the order requisition portal 106 determines if the product 104 is in existing inventory. In some embodiments, the order requisition portal 106, may determine if the healthcare provider 102 has the requisite authority to select the product 104. In this embodiment, the healthcare provider 102 has used the orders requisition portal 106 to build the curated products platform shown. Additionally, the healthcare provider is utilizing the orders requisition portal 106 to request the product 104, such as a prescription.

In some embodiments, the curated products platform 100 may include the discussion module 108. The discussion module 108 is utilized to allow communication between one or more of the healthcare providers 102. For instance, the discussion module 108 may be utilized to allow communication between a physician and a patient. The physician may call the patient to inform the patient of his/her condition then send a message to the patient via the discussion module 108 with a list of products to improve the patient's condition. The discussion module 108 may include communication such as messaging, scheduling, video calling, telephonic calling, group forums, group chats, or other forms of communication.

In some embodiments, the healthcare provider 102 may customize the curated products platform 100 by selecting from a variety of webpage templates. Each template may offer varying services. Different combinations of templates may be available for different layers/levels/areas of the curated products platform 100. A template may include various tiles, ribbons, windows, or the like that can be populated with any of a wide variety of services, features, menus, links, images, content entry forms, text boxes, radio icons, and/or other online products. The healthcare provider 102 may select from a global product library to create the curated products platform 100.

In some embodiments, the curated products platform 100 is more than a customized webpage. The healthcare provider 102 may customize the telemedicine offerings. For instance, the healthcare provider 102 may select to include a wide variety of healthcare services, features, functions, databases, subsystems, and/or other option in the curated products platform 100. The healthcare provider 102 does not create or customize the underlying infrastructure. For instance, the healthcare provider 102 may select face-to-face, live video conferencing. In some embodiments, the healthcare provider 102 may utilize infrastructure provided by a service provider to allow customizing and selecting top-level domains and/or subdomains to make it appear as if the curated products platform 100 is operated by the healthcare provider 102.

Part of the ease of the presently described systems and methods is that the healthcare provider 102 may not need to worry about local data storage, backups, networks, servers to support the software, or the like. The supporting hardware and software for the telemedicine services selected by the healthcare provider 102 may be created, managed, maintained, updated, and/or otherwise cared for without the selecting healthcare provider's 102 knowledge.

In various embodiments, the telemedicine system allows the healthcare provider 102 and/or other providers to customize the curated products platform 100 for their specific practice. For example, a general practice physician may desire to present their “own” curated products platform 100 with a unique interface and a variety of available features and services that is vastly different from the curated products platforms 100 offered by a pharmacist, a therapist, a radiologist, a dietician, a physical therapist, or another healthcare provider 102.

In some embodiments, the telemedicine system allows for modular applications to be selected by the healthcare provider 102 for inclusion in the healthcare provider's 102 customized curated products platform 100. Each of the modular applications may be purchased and/or subscribed to the healthcare provider 102 on an individual basis or as part of packages of products 104 for specific industries.

The discrete modular applications may include third-party applications, interfaces, services, products, and the like that are integrated into the backend of the telemedicine system. In some embodiments, the telemedicine system may act as an integration hub to provide a common or standardized connection between all of the discrete third-party applications, interfaces, products, and services.

The healthcare provider 102 may elect to integrate their curated products platform 100 with any number of custom, standardized, and/or commercially available EMR solutions. In the illustrated embodiment, the service provider provides EMR integration with a wide variety of commercial EMR systems. Such integration may be executed in part using preprogrammed HL7 interfaces or other necessary modes of integration. Integration with alternative standards, such as open EHR and other health record standards, may also be supported. As used throughout, EMR data or an EMR includes or may be substituted by any form of medical, health, personal, financial, or other patient information that pertains to treatments, healthcare, medications, consultations, diagnostics, and the like.

In various embodiments, the telemedicine system may allow existing EMR integration by uploading the EMRs from an existing EMR database to a patient-controlled or physician-controlled account.

Thus, in some embodiments, the healthcare provider 102 may use the telemedicine system to create the curated products platform 100 and may select to include EMR integration with their existing or former EMR system. In such an embodiment, the EMRs from the existing or former EMR system may be accessible and/or imported into the telemedicine system and/or made available within the practitioner-specific curated products platform 100 to the healthcare practitioner and/or their patients.

In other embodiments, a patient may create a patient account on the curated products platform 100. The patient may then use the telemedicine system to upload EMRs and/or request EMRs from the healthcare providers 102. In some embodiments, the patient may select an existing EMR system of a healthcare facility and request that the patient's EMR data be imported into their personal account. In some embodiments, the patient may use the curated products platform 100 to request EMR data from a healthcare facility, and the telemedicine system will contact the healthcare facility electronically or manually to request and ultimately upload the EMIR data of the requesting patient into the patient's account. The patient and/or healthcare facility may pay for the patient's account and storage of EMR data.

Thus, the patient may have independent access to their EMRs through their own personal account. Alternatively or additionally, the patient may have access to his/her EMRs through the curated products platform 100 of his/her healthcare provider 102. In either case, a patient may utilize the curated products platform 100 to access all or portions of his/her medical records, including but not limited to in-person office visit notes, laboratory results, radiological or other study results, medications prescribed, consultation notes, historical data, diagnoses, and/or other EMR data.

In various embodiments, the telemedicine system may allow the healthcare provider 102 to export EMR data for one or more patients to other EMR systems. Thus, notes, messages, pictures, or the like generated by or within the telemedicine system may be exported or shared with other EMR systems that the healthcare facility and/or patient may utilize.

Patients may have access to their EMR data through a personal account that is provided free (or by subscription, per use basis, etc.) by the telemedicine system and/or through one or more of their healthcare provider's 102 curated products platforms 100. In either case, a patient may have access to a “controlled medical record share feature.” The controlled medical record share feature may be utilized to allow patients to control access to their medical record. All or part of the EMR data may be accessible to the patient, and a subset of that data (or all of it) may be shareable by the patient with other healthcare practitioners, insurers, and/or other third parties. For example, a patient may be able to control the access privileges of visit notes, lab results, radiology studies, etc. In various embodiments, the entire record can be shared, or selective parts of the EMR may be shared. The patient may also rescind access to those parts of the medical record at any time. Thus, the proposed systems and methods give patients unprecedented control over their personal EMR data to share and rescind access to any third-party.

In some embodiments, if the patient elects to share EMR data via the telemedicine system with a healthcare practitioner that is not a telemedicine system member, the telemedicine system may contact the healthcare practitioner out of the telemedicine system (e.g., via email, phone, text, letter mail, etc.) and provide an option for one-time secure viewing of the EMR and/or invite the healthcare practitioner to create an account for one-time or continuous access to the shared EMR. As previously described, fees may be charged to any of the parties involved for uploading, viewing, sharing, access, and/or the other features and services provided by the system.

The service provider system may actually store the EMR data or may act as a portal to access EMR data stored in other EMR systems managed by individual healthcare providers 102 or third parties. Thus, a first physician may use the telemedicine system to access EMR data of a patient where the EMR data is stored on the telemedicine system, where the EMR data stored in a database managed by the first physician, where the EMR data is stored in a database associated with another EMR system, where the EMR data is stored in a database managed or associated with a second physician, and/or where the EMR data is stored in a database managed by the service provider (e.g., in a situation in which a patient uploaded medical documents/files to the system).

In some embodiments, a patient medical record share portal may show medical records of the patient with dates, provider, specialties, practices, reasons for visits, and the current number of times or people with whom the medical record has been shared. Selecting the share count may allow the patient to manage the sharing privileges relating to that particular medical record.

In various embodiments, a patient may share EMR data with healthcare providers 102 who are subscribers or members of the telemedicine system and/or with healthcare providers 102 who are not members or subscribers. For example, in one embodiment, the patient may enter an email or telephone number of the physician who is not a subscriber to the telemedicine system. The telemedicine system may then contact the physician using the telephone number and make them aware that EMR data has been shared. The physician may download or otherwise be provided with access to the EMR data and/or may be invited to become a permanent or temporary subscriber.

In various embodiments, patients or other users of the telemedicine system may be able to securely share EMR data with any other person (not just healthcare providers 102) by entering contact information that the telemedicine system will use to contact the intended recipient. As a specific example, if the patient recently received an ultrasound of a baby, that ultrasound may be part of an EMR and accessible by the patient within the telemedicine system. The patient can choose to share the ultrasound with any number of people by simply entering the contact information of the intended recipients. In various embodiments, the patient may elect to share the ultrasound in a secure environment (e.g., within the discussion module 108 or other portal associated with the telemedicine system) or outside of a secure environment (e.g., within the discussion module 108 via an unsecured email or MMS message).

The telemedicine system may provide a list of practitioners within a network known to the patient, entered in the telemedicine system, and associated with a particular healthcare facility, current subscribers to the telemedicine system, and/or another list of healthcare providers 102. The telemedicine system may also provide a list of “current shares” and allow the patient to rescind the sharing of the particular medical record with respect to one or more of the “current shares.”

Whether through an independent personal account or through one or more of their healthcare provider's 102 curated products platform 100, patients may have access to and control of their radiology study and associated medical records via a personal radiology study and medical record storage suite. In some embodiments, healthcare providers 102 may be charged for maintaining a database of medical records and/or radiology images/studies.

The personal radiology study and medical record storage suite may allow the patient to control access to their radiology studies and associated records. In one embodiment, patients may have the ability to request their radiology or other studies (ultrasound, echocardiograms, etc.) and have them uploaded to the telemedicine system or to have them made available via a short-term or long-term portal.

There may be a one-time or subscription-based fee charged to the healthcare provider 102. Any of a wide variety of financial models might grant access to the study for an unlimited (or limited) amount of time. All or part of the studies and associated images, graphs, measurements, or the like may be accessible to the patient, and a subset of that data (or all of it) may be shareable by the patient with other healthcare providers 102, insurers, and/or other third parties.

In some embodiments, if a patient elects to share the data with a member healthcare practitioner, the member healthcare practitioner may receive a notice that the data has been made available and access it via a corresponding portal. If the healthcare practitioner with whom the data has been shared is not a member, the telemedicine system may contact the healthcare practitioner (e.g., via email, phone, text, letter mail, etc.) and provide an option for one-time secure viewing of the EMR and/or invite the healthcare practitioner to become a member for continuous access to the shared EMR. As previously described, fees may be charged to any of the parties involved for uploading, viewing, sharing, access, and/or the other features and services provided by the system.

In some embodiments, a login and signup portal for the healthcare provider 102 may be utilized to allow for the healthcare provider 102 to sign in or sign up for a secure account that is HIPPA compliant.

In some embodiments, a patient or a prospective patient may utilize the telemedicine system to select any healthcare provider 102, including healthcare providers 102 unaffiliated with the curated products platform 100. In some embodiments, adoption of the curated products platform 100 is encouraged by allowing patients and prospective patients to select, contact, and/or be connected with any healthcare provider 102, even those healthcare providers 102 that are not affiliated with the curated products platform 100. In such embodiments, the unaffiliated healthcare provider 102 may be encouraged to become an affiliate.

In some embodiments, free consultations may be offered to entice new customers or retain existing customers. In some instances, consultations that would normally cost money may be offered for free or at a discounted price if selected in the context of purchasing the product 104. For example, the purchase of particular face cream or subscription to medication may include a free telepresence consultation. Such a consultation may also be required for the purchase. For instance, a prescription medication available for purchase may be coupled to a telepresence consultation utilized to allow the healthcare provider 102 to provide the requisite prescription for the medication.

In some embodiments, individuals may be assigned various roles with the curated products platform 100. In some embodiments, appointment types may be customized and configured according to various embodiments. In some embodiments, coupons may be created for various products 104. In some embodiments, the products 104 may be customized.

The curated products platform 100 may be utilized to combine e-commerce with a variety of professional service industries. Various consultations may be offered by the healthcare provider 102 via the curated products platform 100. Integration of telemedicine and/or other consultation services may be integrated as part of a concierge offering of an existing website of a business using the telemedicine system for backend support. In some embodiments, the curated products platform 100 is used to configure a concierge offering for existing businesses. For example, the curated products platform 100 can be customized in a matter of minutes for integration with an existing website to provide a concierge package of products 104 that may be customized for the particular business.

Various consultation offerings may be supported via the telemedicine system integrated into an online concierge offering. Any of a wide variety of tiered, discounted, incentive-based, and other financial models may be used. For example, in some embodiments, a variation of a concierge subscription model may be used where the patient pays the healthcare provider 102 on a monthly or yearly basis for predetermined telemedicine and/or in-person services.

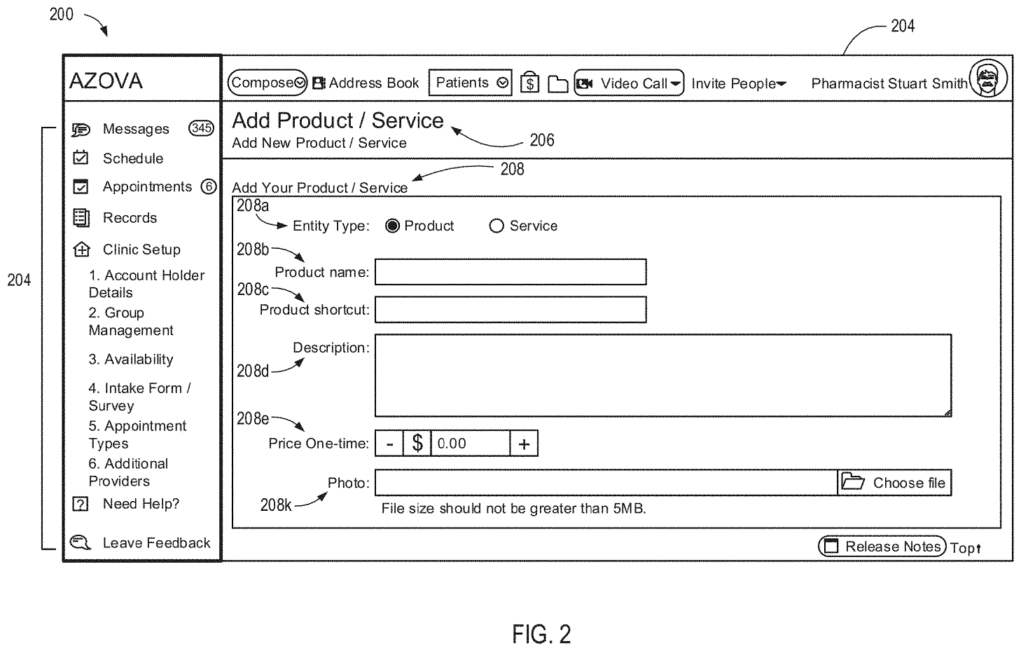

FIG. 2 illustrates a screenshot of a possible embodiment of a GUI for selecting a product 204 from a curated products platform 200 via an orders requisitions portal 206. The orders requisitions portal 206 may include a healthcare provider 202 and the one or more products 204. In some embodiments, the orders requisitions portal 206 may include an order form 208 and an order request. The order request contains the data gathered from the order form 208. The orders requisitions portal 206 is utilized to allow the healthcare provider 202 to request products 204. The requested products may be displayed as the plurality of products and/or the subset of products. In some embodiments, the healthcare provider 202 selects the products 204 by populating the order form 208. The order form 208 may include an entity type field 208a, a product name field 208b, a product shortcut field 208c, a description field 208d, a price one-time field 208e, and a photo field 208f. In some embodiments, the order form 208 may include more fields. In some embodiments, the order form 208 may include fewer fields.

In some embodiments, the entity type field 208a may be utilized to detail the type of product 204 being requested. In this example, the healthcare provider may select via bubbles either an entity type product or an entity type service. The product name field 208b allows the healthcare provider 202 to name the requested product. In some embodiments, the healthcare provider 202 may populate the product name field 208b by typing a custom name or by choosing from a menu of names. The products shortcut field 208c may populate as the healthcare provider 202 fills the field. For example, the healthcare provider 202 may start typing the requested product 204 name and after two letters are typed options are provided to the healthcare provider 202 for potential product 204 names. The healthcare provider 202 may select a name from the list. The description field 208d may be utilized to describe the product 204 requested. In some embodiments, the description field 208d may be populated by the healthcare provider 202 typing and/or handwriting a description. In some embodiments, the description field 208d may be populated by a PDF, JPEG, or similar document. The price one-time field 208e may be utilized to provide a price for the product 204. In this embodiment, the price one-time field 208e may be populated by typing a price. In some embodiments, the price one-time field 208e may be populated by selecting bubbles, buttons, or other GUI. In this embodiment, the price one-time field may be populated by selected an increase or a decrease button to increase or decrease the displayed price. The photo field 208f is utilized to upload a photo. The photo may be of the requested product 204.

The requested products 204 are added to the healthcare provider's 202 curated products platform 200. In some embodiments, the curated products platform 200 may display a mini-store, online store, the subset of products, or other display. In some embodiments, the orders requisitions portal 206 determines if the selected product 204 is available for purchase. Determining if the product 204 is available for purchase may include determining if the product 204 is in stock or otherwise available for shipping, if the healthcare provider 202 requesting the product 204 is eligible to display or purchase the products 204, or for any other related reason. In some embodiments, the order request is sent to the telemedicine system to determine the availability of products 204.

In some embodiments, the healthcare provider 202 selects the products 204 for the curated products platform 200, where the products 204 may include: urgent messages, urgent telephone call visits, insurance authorization requests, insurance notification features, after-hours patient request management and forwarding, physician's note management, generation, forwarding, request, etc., secure instant messaging, secure text messaging, secure MMS messaging, secure SMS messaging, secure email, secure out-of-band messaging, secure messaging through a third-party or other secure healthcare-specific messaging application, and/or any other service or product provided by a healthcare practitioner, practitioner's assistant, billing administrator, insurance administrator, and/or other involved party whose service or product can be provided or at least partially provided in an online format via a digital health platform. In some embodiments, the discussion module may utilize one or more of the products 204 to allow for communication. For instance, messages may be both the requested product 204 and a portion of the discussion module.

The healthcare provider 202 may customize the curated products platform 200 by selecting categories, manufacturers, and/or individual products 204 to add to the curated products platform 200. In some embodiments, selecting a brand-name version of the product 204 for the curated products platform 200 automatically results in including a generic version of the product 204. In some embodiments, the product 204 is added to the curated products platform 200 via the orders requisitions module 206.

The curated products platform 200 associated with the healthcare provider 202 may appear to be managed and run by the healthcare provider 202, when, in reality, the product 204 on the curated products platform 200 may be managed and shipped by the service provider. Profits may be shared according to any of a wide variety of profit sharing sales approaches commonly used in the industry.

In some embodiments, the product 204 may be selected by selecting a category and/or manufacturer. In some embodiments, the manufacturer and/or category classifications may be carried through into the curated products platform 200. Alternatively, the classifications may be removed or customized by the healthcare provider 202.

The product 204 may be fully or partially customized by the healthcare provider 202 to conform to, for example, physician's particular practice. In some embodiments, the product 204 may include customizable features that are readily customizable by the healthcare provider 202. In other embodiments, the healthcare provider 202 may customize the product 204 by requesting a programmer from the service provider and/or a third-party programmer delete, add to, supplement, and/or otherwise modify features, advantages, or aspects of the product 204.

In some embodiments, the telemedicine system may allow a pharmacist to configure a dashboard to allow for medication therapy management (MTM) visits and to facilitate telemedicine visits with other associated healthcare providers 202. This facilitation “visit” type allows the pharmacist to charge in exchange for taking the time to help a patient get care with distant or remote healthcare providers 202.

In such an embodiment, the pharmacist's curated products platform 200 may be utilized as “Online Clinic,” may include the product 204, and/or an “MTM visit,” for which the pharmacist may bill the patient's insurance, the patient, and/or the associated healthcare provider 202.

The pharmacist's online clinic may also include a “Help with an Online Visit” product 204 that directs the patient to a page that explains that the pharmacist can help them to use technology to see any healthcare provider 202 in their state who subscribes to the telemedicine system. The pharmacist may set a fee for this type of help/visit/facilitation. Once the patient has paid for this visit (automatic billing may bill the patient later), the patient may then select the healthcare provider 202 available via the telemedicine system by entering the handle of the healthcare provider 202.

Once redirected to the healthcare provider's 202 curated products platform 200 interface, the patient may pay for (or not, depending on the patient's benefits etc.) the visit and proceed to obtain a telemedicine consultation with the healthcare provider 202 (e.g., a physician) in conjunction with the assistance of the pharmacist or his/her staff member.

In various embodiments, when a patient selects “MTM visit” a form may be presented to capture requisite or useful data for the pharmacist to conduct the MTM visit. The curated products platform 200 can be integrated with drug, food and/or vitamin/supplement interaction monitoring software. The interface may help the pharmacist conduct an efficient and thorough MTM visit.

The telemedicine system may also provide an interface with medical supply companies, such as durable medical supply (DME) companies, diabetic supply companies, continuous positive airway pressure (CPAP) supply companies, Orthopedic supply companies, and/or other medical supply companies.

The telemedicine system may provide discounts on diabetes supplies, CPAP supplies, DME, and orthopedic supplies. Similarly, the telemedicine system may provide discounts on prescriptions drugs. In some embodiments, a telemedicine visit may be preceded by a direct recommendation for products 204 on one dashboard and/or as the direct result of a telemedicine consultation. Thus, the telemedicine system may drive demand and/or increase awareness of patients for particular products or services.

In some embodiments, the telemedicine system may implement a bidding process to the DME or other supply companies to be presented to the healthcare provider 202 that selects one of the buttons for the diabetes supplies, CPAP supplies, DME, and orthopedic supplies. The bidding process may be used to provide the best price to the purchasing healthcare provider 202 and/or to maximize the percentage collected by the telemedicine system.

The telemedicine system can be adapted for any of these industries to do “Online Specials” in conjunction with an in-person face-to-face video consultation or a store and forward or other online or digital consultation type (including telephone). The On-Call Button can also serve as an answering service for these businesses.

The healthcare provider 202 may customize their curated products platform 200 to include a list of laboratories and/or imaging centers from which a patient may import electronically (e.g., via formal or simplified order requisition of medical records) medical information (e.g., pathology test results, images from a medical imaging consultation, etc.). In some embodiments, the laboratories and/or imaging facilities selected by the healthcare provider 202 may appear on the healthcare provider's 202 dashboard during a patient consultation. The healthcare provider 202 may schedule consultations, tests, imaging, etc. with any of the listed laboratories and/or listed imaging facilities.

In various embodiments, a dashboard may allow a physician, an assistant, a patient, and/or a patient's representative to schedule an e-visit, an in-office appointment, a house call, and/or other consultation. In one possible embodiment, scheduled visits (whether e-visit, in-office, house call, or other) may be color coded on a calendar.

The healthcare provider 202 may offer one or more types of visits and online scheduling of the same. In some embodiments, the healthcare provider 202 may use the telemedicine system to schedule in-person or e-visits on a case-by-case basis.

For instance, a Find an Appointment or Find a Provider page may allow a patient to select the type of visit, specialist needed, an address, identifying information, medical history, medically relevant facts, maps, contact information, etc. that can be used to schedule a first or follow-up visit. The specific healthcare provider 202 may be presented to the patient for selection along with available appointment times and scheduling abilities.

In some embodiments, the healthcare providers 202 may be presented with or search for specials, membership opportunities, package deals, and/or concierge service. For example, a patient may visit Laboratory A for a blood test and then visit Laboratory B for a pathology test. The patient may then return to the healthcare provider 202 for a follow-up consultation. The healthcare provider 202 may access the dashboard during the follow-up consultation and open an order request to select and/or request the desired tests or studies from Laboratory A and Laboratory B.

As described herein, the telemedicine system may facilitate scheduling patients for e-visits, in-office visits, in-home visits, etc. Additionally, the telemedicine system may allow for integration with population health management programs. Population Health Management (PHM) may be understood as an aggregation of patient data across multiple health information technology resources. PHM may also include the analysis of the aggregated data into a single, universally available patient record. PHM may also include the actions through which the healthcare providers 202 may improve both clinical and financial outcomes. A goal of PHMs is often to improve both care and financial efficiency.

In various embodiments, the telemedicine system provides integrated access to healthcare providers 202 and other entities associated with a capitated plan model or an accountable care organization (ACO). Such entities may need to monitor large numbers of patients with, for example, certain chronic diseases such as asthma, diabetes, hypertension, congestive heart failure, and/or chronic obstructive pulmonary disease (COPD). The telemedicine system may provide (1) remote monitoring capabilities, (2) the ability to schedule any number of patients with any number of common problems, and all in conjunction with (3) scheduling of any number of other visit types using the same curated products platform 200.

Because the telemedicine system in many embodiments is not restricted to any particular electronic medical type or format, the healthcare providers 202 can configure their preselected platform 200 to interface (e.g., via a dashboard interface) with any of a wide array of (potentially all) laboratories, imaging centers, and other healthcare-related facilities. In some embodiments, the order request may be electronically sent and received or may be sent and received manually (e.g., hardcopy), depending on the capabilities of the selected laboratory, imaging center, and/or another healthcare-related facility.

In some embodiments, custom HL7 interfaces for each EMR may be avoided or eliminated by integrating each facility with the telemedicine system, regardless of the specific EMR format utilized. Based on participation and integration, the telemedicine system may allow for any healthcare provider 202 to access data from any laboratory, imaging center, and/or healthcare-related facility in the world.

Underlying interfaces of the telemedicine system with order requests may be done in some embodiments via a digital version of the order form 208 hosted on the curated products platform 200. In other embodiments, the underlying interface of the telemedicine system for order requisitions may include an interface with the order requisition portal 206 on a website or other portal of the laboratory, imaging center, or another healthcare facility.

When interfacing with the electronic requisition portal of an external laboratory, imaging center, or another healthcare facility, the telemedicine system may provide the necessary data to complete the order request. Such data may vary based on the specific electronic order requisition portal 206 and may include patient name, date of birth, address, insurance information, billing address, shipping address, electronic contact information, other demographic information, personal identification numbers, and/or the like.

In various embodiments, the healthcare providers 202 including patients, healthcare practitioners, laboratories, imaging centers, and/or other healthcare entities/facilities may have unique handles that allow for secure messaging and interfacing via the discussion module without potentially revealing personal information between entities. That is the telemedicine system may keep, hide, or selectively reveal private, personal, HIPPA protected, and/or other information between interacting entities. For example, the healthcare providers 202 may communicate using secure messaging within the telemedicine system using specific handles via the discussion module 106.

In some embodiments, a laboratory may have access to a HIPPA secure dashboard within the telemedicine system where they can manage orders made via the telemedicine system. Alternatively, orders requested via the telemedicine system may be transmitted to the relevant laboratory via email, e-fax, or physical mail. In various embodiments, the telemedicine system may provide the laboratory an interface whereby they can send invoices directly to the requesting healthcare provider 202 using the handle of the healthcare provider 202, an associated insurance, and/or other healthcare provider 202. The invoice may be sent to an address (electronic or physical) associated with the handle.

In some instances, a custom order request may be built for each laboratory that, when selected from the healthcare provider's 202 curated products platform 200, may deploy the order request with the healthcare provider's 202 demographics and insurance information as required by the laboratory. Each product 204 that is offered by the laboratory may be listed with a respective price and description as well as any requisite test kits that may be needed to collect that specimen that is ordered by the healthcare provider 202.

The listing may be integrated with an online shop or marketplace of the telemedicine system where the respective laboratory will have a backend account to upload all products, their descriptions, and any required testing kits or supplies that will be needed for that particular test. The cash price, discount price based on membership, insurance price, wholesale price, or other price for the test and/or supplies may also be listed. When the healthcare provider 202 selects a particular test while an appointment page is open in their dashboard, the test may be sent to the patient's chart as a “healthcare practitioner's order” along with a link to purchase the test. The patient may also indicate via a button, menu selection, or added note that the laboratory should bill an insurer.

Laboratory interfaces on the telemedicine system may provide an indication of specimen collection types to the healthcare provider 202. Collection types might include:

(1) Remote phlebotomy services, in which case the patient may be presented with a link to purchase a remote phlebotomy service;

(2) In-home saliva, dried urine, finger stick, tissue collection, and blood spot tests, in which case the patient may be presented with a link to purchase the blood work. Once payment has been received by the telemedicine system or an associated payment system, the telemedicine system may route the order to the respective laboratory. The laboratory may then ship the appropriate test kit(s) to the patient. Once the results are available, the laboratory may upload and transmit the results to the patient and/or the physician via the curated products platform 200 (e.g., via an entity-specific dashboard) by using the handle of the healthcare provider 202.

(3) Visitation to a blood draw station or other specimen collection site, in which case the actual requisition is sent to the patient as a document. The patient can print it out and take it to the blood draw station or another specimen collection site.

As indicated above, laboratories, imaging centers, and/or other healthcare-related facilities may sell, advertise, and/or otherwise offer products and/or services via the curated products platform 200, such as within a website. When the healthcare provider 202 creates the customized or semi-customized curated products platform 200, the healthcare provider 202 may select (or it may be automatically included) to create an associated e-commerce interface.

For each laboratory, imagining center, and/or other healthcare-related facility included by the healthcare provider 202 on the customized preselected product platform 200, the products 204 may be included automatically within the personalized e-commerce interface. Products 204 for each laboratory, imagining center, and/or another healthcare-related facility may be associated with a SKU allowing for easier inclusion and unique identification on each unique e-commerce interface of a plurality of the healthcare provider's 202 customized, curated products platforms 200.

Laboratories may upload a list of the products 204, along with associated costs and details of administration via an e-commerce interface. The laboratories may indicate the name of the laboratory, payment types accepted, insurances accepted, collection method for the test, test type, etc. For each product 204 offered, the MSRP or standard pricing of the lab test may be indicated. In some embodiments, the listed MSRP price may be required to be less than, equal to, or greater than (but capped at, for example, 10% greater than or 20% greater than) other online prices offered by the laboratory. A sale price for telemedicine system users may be listed as well (e.g., 20% below MSRP or standard pricing).

Examples of collection methods may include blood draw stations, mobile phlebotomy, dried urine, cheek swab, saliva, culture swab, finger stick, tissue specimen, and the like. Examples of test types include blood, urine, tissue, genetic, and the like.

In various embodiments, when the healthcare provider 202 (e.g., a primary care physician) selects a laboratory, imaging facility, or another external healthcare facility from the telemedicine system, the order form 208 may be populated with all or a subset of the tests that can be ordered from the selected laboratory, imaging facility, and/or other external healthcare facility. For example, all of the available tests may be populated on the order form 208 in alphabetical order and/or in another order based on customized preferences of any involved entity.

Multiple possible scenarios are possible when the healthcare provider 202 orders particular lab work from a laboratory (similar scenarios are possible for imaging centers and/or other entities). Examples of a few are provided below in which the healthcare provider 202 is referred to as a “provider”:

Scenario 1: The test is cash only at a standard blood draw station. The test may be sent to the patient in their Plan section (of their chart) with a button (or other icon or option) to purchase. When the patient pays, the patient will receive the order form 208 as a receipt. The order form 208 may comprise relevant patient demographics and/or other personal information, ordering/requisitioning provider demographics and/or personal information, the test(s) ordered, an indication of where the results should be sent (e.g., to the patient and/or the requisitioning provider), the physician's electronic signature, and/or the patient's and provider's (e.g., the physician's) handles. In various embodiments, the order form 208 may look like a standard order form. In some embodiments, the order form 208 may include a UPC code or other electronically readable identification information that will allow electronic tracking and/or order history information.

Scenario 2: The test is cash only and is dried urine, cheek swab, blood spot, finger stick, or the like. The provider may select one or more of these tests; the respective tests may then appear in the plan section of the patient's chart along with a button to purchase the products 204. The order form 208 may include of patient demographics/information, ordering provider's demographics/information, the test(s) ordered, an indication if the results should go to the patient and/or the provider, the physician's electronic signature, and the patient and provider's respective handles. When the patient purchases the products 204 (i.e., tests), the order request may be sent to an appropriate laboratory's dashboard and display as “pending orders.” The laboratory may then ship the test kit to the patient or have the test kit ready for pickup by the patient. The patient may then collect the specimen and send it back to the lab. When the results are ready, the lab may upload them to the messaging platform via the discussion module and send them to the patient and/or the provider using their respective handles.

Scenario 3: The lab test is cash only and is mobile phlebotomy. The mobile phlebotomy may be sent as a separate line item to the patient for purchase. The provider may then order the mobile phlebotomy.

Scenario 4: The test is insurance eligible and is mobile phlebotomy. The patient may receive the order form 208 as well as an appropriate laboratory. The laboratory may then contact the patient to arrange for phlebotomy and insurance billing.

Scenario 5: The test is insurance eligible and at a standard blood draw station. The patient may not need to pay to get the order form 208. The order form 208 may be embedded/attached to the visit note from the provider. The patient can print it or take it to the appropriate laboratory for blood work.

Scenario 6: The test is insurance eligible and is dried urine, cheek swab, blood spots, saliva, finger stick, and/or tissue collection. The order form 208 may be sent to the patient and the laboratory. The laboratory may then send the collection kit to the patient and manage the insurance billing information.

Scenario 7: The test is cash only and tissue collection. The product 204 may be sent to the patient's plan section of their note from the provider for purchase. The patient may pay for the test, and the order may then be transmitted to the lab. The lab may then send the ordering provider the test kit (or the patient can go to the provider's office where the provider may already have some of the test kits), and the tissue specimen will be taken and sent to the lab. An example of this scenario may be when a tissue pathology laboratory is used to process specimens that are taken in a provider's office, but that was ordered by the provider as the result of a telemedicine visit.

In various embodiments, under each lab product entered to the site, there may be a box for “Provider's Comments to the Laboratory.” This may appear along with the product and/or service that will be in the plan section of the patient's note once the provider has selected the product 204. Accordingly, a provider can specify/clarify details of the test/service for the laboratory.

In some embodiments, when the healthcare provider 202 configures the laboratories from which lab work will be ordered, the healthcare provider 202 may simply select (e.g., via a click) those laboratories from a list of available laboratories that the healthcare provider 202 wishes to utilize. The selected laboratories may be used to populate the healthcare provider's 202 dashboard under “Laboratories.”

Radiology may be very similar or the same as the examples and scenarios described herein with regard to laboratories. For example, order requests may be cash or insurance-eligible and the order requests may be sent to the patient and/or the imaging center. The telemedicine system may include all order requests, a place for patients to consent to have their results released to themselves and/or to the requisitioning healthcare provider 202.

In one specific example, a GUI is configured to allow the healthcare provider 202 to select products 204 from a drop-down menu of a product library of available products 204. The drop-down menu may include: live face-to-face video visits, store-and-forward visits, telephone visits, urgent telephone visits, prior authorization services, prescription drug price comparisons, medical procedure and office visit price comparisons, physician's note services, and secure text visits. As indicated to the right of the drop-down menu, cost and/or unit pricing options may be selected.

In some embodiments, the healthcare provider 202 may elect to include the products 204 that allow cosmeceutical visits. In such an embodiment, the practitioner specific curated products platform 200 may allow a patient to conduct a virtual office visit (real-time, store-and-forward, and via telephone, secure messaging, or other methods for communication) for the primary or sole purpose of obtaining a prescription, such as a prescription-strength cosmeceutical product. The telemedicine system may allow the healthcare provider 202 to issue the prescription and, in some embodiments, initiate delivery of the prescription through the telemedicine system, the healthcare provider's 202 mini-store, and/or a third-party prescription vendor.

The healthcare provider 202 may further select to include “store-and-forward visits” and “face-to-face video visits” within their customized or individual curated products platform 200. The healthcare provider 202 may select to charge $99 per store-and-forward visit and $149 per face-to-face visit. In some embodiments, additional customization of the pricing models may be available. In some embodiments, integration with insurance companies for medical billing may be available.

Once the healthcare provider 202 selects the desired products 204 from the drop-down menu representative of the product library, the selected products 204 may be added to the curated products platform 200 (e.g., they may be added as selectable icons to a practitioner-specific portal or webpage).

In various embodiments, the product library may include various products 204 that provide for and/or relate to patient medical records. In some embodiments, the healthcare provider 202 may elect to include integration features in their curated products platform 200 that are configured to facilitate electronic medical record (EMR) integration within one or more existing EMR systems.

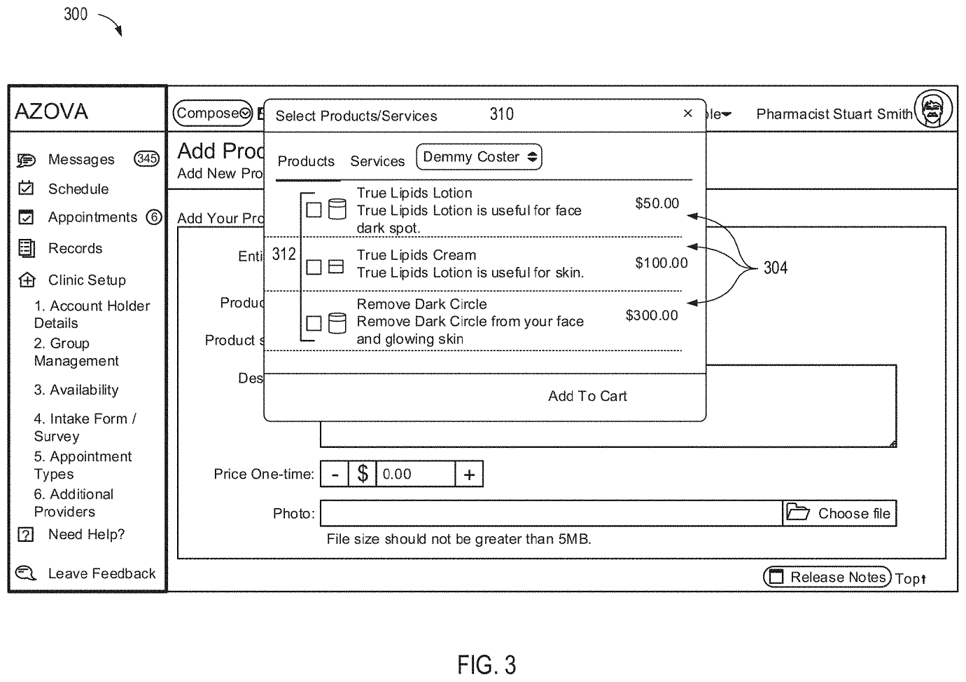

FIG. 3 illustrates a screenshot of one possible embodiment of a GUI of a subset of products module 310 utilized to display a subset of products 312. The subset of products module 310 may store data related to the subset of products 312. In some embodiments, the subset of products 312 is selected by the healthcare provider from a curated products platform 300 displaying a plurality of products from a product library. For instance, a healthcare provider, such as a physician, may select at least one product 304 for a patient from the preselected product platform 300. The order requisitions portal may receive the selected products. In some embodiments, the order requisitions portal may display the subset of products module 310 to the healthcare provider (i.e., a patient). In some embodiments, the subset of products module 310 may be displayed as link, button, or other entity. The healthcare provider may click or otherwise select the subset of products module 310 to enable the subset of products 312 to be displayed. In some embodiments, the healthcare provider may click on the subset of products module 310 and the subset of products 312 may be automatically purchased. The at least one healthcare provider, the patient, may select the product 304 from the subset of products 312. The healthcare provider, (or the patient), may purchase and/or select the products 304 directly from the subset of products module 310.

The subset of products module 310 is utilized to allow the healthcare provider to select and/or purchase products 304 directly from the subset of products rather than a large group of products. The subset of products is utilized to allow the healthcare provider to select the products 304 relating to each specific healthcare provider. For instance, the subset of products 312 chosen by a physician may be different for the patient with a broken leg and the patient with the flu. In some embodiments, the healthcare provider (or a patient) may receive the subset of products module 310 with the subset of products 312. The healthcare provider may select and/or purchase products 304 from the subset of products 312. In some embodiments, the patient may purchase the products 304 directly from the subset of products module 310 (i.e., a link). Purchasing directly from the subset of products module 310 cuts time spent looking for the correct product 304 and ensures purchase of the correct product 304.

For instance, a physician may meet with a patient and recommend that the patient use a particular soap and lotion combination as a skin treatment for a certain time period. The physician may conduct the visit via a teleconference visit through the preselected product platform 300 associated with the physician and then provide a treatment summary utilizing the discussion module via a secure messaging application. The treatment summary may include the subset of products module 310 that is utilized to allow the patient to purchase the recommended skin treatment products from the preselected product platform 300 associated with the physician. The subset of products module 310 may utilize previously stored financial and/or shipping data, such that a single click (e.g., one click), which may be all that is required to complete the order of the skin or other healthcare treatment products.

The master list of the products that may be included by selection in the healthcare provider's preselected product platform 300 may include any of a wide variety of healthcare or other items, such as, but not limited to: bandages, medical supplies, medical equipment, skin care, personal care, supplements, medications, treatment plans, educational material, books, soaps, lotions, personal hygiene items, foot care items, incontinence items, hair care, tests, monitoring equipment, lip care, feminine care, therapeutic devices, hot pads, ice packs, etc.

For instance, the preselected product platform 300 for a healthcare facility may include the products 304 that are accessible to the physicians, nurses or other staff associated with the healthcare facility. The curated products platform 300 may include supplies, clothing, medical devices, and/or other equipment commonly used by healthcare practitioners.

In some embodiments, the healthcare provider (e.g., healthcare facility) may utilize the service provider's system to track inventory and usage of supplies and equipment by the healthcare provider (e.g., physicians, nurses or other staff) associated with the healthcare facility. The telemedicine system may allow the healthcare provider to “pay” for items on an account basis that simply provides for internal monitoring. Purchases made under the system may be shipped by the telemedicine system or simply routed for internal shipping to a supply manager of the healthcare provider.

The telemedicine system may allow for the integration of a telemedicine visit into the product description page of any of the products 304. For example, the marketplace described herein may include the products 304 dispensed by a physician only. These products 304 may require a physician recommendation, code, or prescription. A link to the healthcare provider's telemedicine clinic may be displayed on the product page so that patients/shoppers can select it and get the appropriate recommendation for the product 304 (potentially via a telemedicine visit with a physician or pharmacist). The product 304 “buy buttons” may trigger a pop-up that indicates that the product 304 requires a physician (or another provider) code, recommendation or prescription and/or initiates the proper telemedicine visit.

Such links and notices may be provided anywhere within the marketplace to prompt a perusing customer to get a consultation to determine if the particular product 304 is right for them and/or to give the healthcare provider the opportunity to close the sale and/or to upsell.

A system may include a network communication module (e.g., ethernet, wifi, optical connections, etc.) that connects server computer devices to remote, client computer devices. The system may initiate healthcare consultations in response to a request to purchase a healthcare product. That is, the purchase and telemedicine consultation of a product, such as a product requiring a prescription, may be initiated by the selection of a product for purchase by a consumer. Thus, rather than a patient seeking treatment via a consultation that results in a physician-prescribed product, the presently described systems and methods contemplate a scenario in which a consumer (not yet a patient, but can be referred to as a patient since they will become a patient) wants to purchase a product that requires a prescription. In response to the consumer request to purchase the product, the system initiates a telemedicine consultation according to a consumer-directed process. Specifically, a user interface module (e.g., instructions executed by a processor) provides a patient user interface (UI) to a first client device enabling a patient using the first client device to view and selectively purchase healthcare products available for shipping to the patient that require a prescription from a healthcare practitioner enrolled in the system.

A consultation offering module (again, implementable as instructions executable by a processor on a server or client device) presents a graphical user interface to the patient with at least two different consultation visit types suitable for the patient to obtain the prescription for the selected product. The system then receives a selection of a consultation visit type by the patient via the graphical user interface. The system then presents, via the graphical user interface, available healthcare practitioner types that offer the patient-selected consultation visit type. The system then receives a selection of a type of healthcare practitioner by the patient. The system then presents the patient with list of healthcare practitioners enrolled in the system that (i) have authority to provide the prescription, (ii) offer the patient-selected consultation visit type, and (iii) are the patient-selected type of healthcare practitioner.

The system may include a selection module (instructions executed by a processor of a server or a client computing device) that receives, from the patient, a selection of one of the presented healthcare practitioners. A consultation module (instructions executed by a processor of a server or a client computing device) then initiate a healthcare consultation of the selected consultation visit type between the patient using the first client device and a healthcare practitioner of the patient-selected type of healthcare practitioner using a second client device.

A prescription generation module (instructions executed by a processor of a server or a client computing device) enables the consulting healthcare practitioner to generate a prescription in connection with a completed consultation to enable the patient to purchase at least one of the products that require a prescription. A shipping module (instructions executed by a processor of a server or a client computing device) facilitates shipping the purchased product to the patient.

In various embodiments and/or in combination with any of the other embodiments described herein, the service provider may allow corporations, employers, insurance companies, and/or other groups to form wellness communities. These communities can utilize the healthcare providers who are contracted with and/or employed by the telemedicine system. Alternatively, the wellness communities can utilize their own physicians or other independent physicians. Any or all parties involved may utilize any or all of the software solutions described herein.

The healthcare provider may customize their mini-store by selecting categories of products 304, manufacturers of products 304, and/or individual products 304 that they would like to add to their mini-store. In some embodiments, the inclusion of a brand-name version of the product 304 in the mini-store may automatically result in the inclusion of a generic version of the same product 304.

In some embodiments, products 304 may be selected by narrowing down the category and/or manufacturer first. In some embodiments, the manufacturer and/or category classifications may be carried through into the healthcare provider's mini-store. Alternatively, the classifications may be removed or customized by the healthcare provider.

The mini-store presented on the healthcare provider's practitioner-specific curated products platform 300 may look like it is managed and run by the healthcare provider, when, in reality, the products 304 may be managed and shipped by the service provider. Profits may be shared according to any of a wide variety of profit sharing sales approaches commonly used in the industry.

As a specific example, a physician may meet with a patient and recommend that the patient use a particular soap and lotion combination as a skin treatment for a certain time period. The physician may conduct the visit via a teleconference visit through the physician's curated products platform 300 and then provide a treatment summary via a secure messaging application via the discussion module. The treatment summary may include the subset of products module 310 that allows the patient to purchase the recommended skin treatment products from the physician's mini-store. The subset of products module 310 may utilize previously stored financial and/or shipping information, such that a single click (e.g., one click) may be all that is required to complete the order of the skin or other healthcare treatment products.

The master list of products that can be included by selection in the healthcare provider's mini-store may include any of a wide variety of healthcare or other items, such as, but not limited to: bandages, medical supplies, medical equipment, skin care, personal care, supplements, medications, treatment plans, educational material, books, soaps, lotions, personal hygiene items, foot care items, incontinence items, hair care, tests, monitoring equipment, lip care, feminine care, therapeutic devices, hot pads, ice packs, etc.

In some embodiments, a healthcare facility may include a mini-store of items that are accessible to healthcare providers associated with the healthcare facility. Such a mini-store may include supplies, clothing, medical devices, and/or other equipment commonly used by healthcare providers.

In such an embodiment, the healthcare facility may utilize the telemedicine system to track inventory and usage of supplies and equipment by internal healthcare practitioners. Such a system may allow healthcare practitioners to “pay” for items on an account basis that simply provides for internal monitoring. Purchases made under such a system may be shipped by the telemedicine system or simply routed for internal shipping to a supply manager of the healthcare facility.

The telemedicine system may allow for the integration of a telemedicine visit into the product description page of any product or service. For example, the marketplace described herein may include products 304 that are physician-dispensed only products 304. These products 304 may require a physician recommendation, code, or prescription. The subset of products module 310 to a providers' telemedicine clinic may be displayed on the product page so that patients/shoppers may select it and get the appropriate recommendation for the product 304 (potentially via a telemedicine visit with a physician or pharmacist). The product 304 “buy buttons” may trigger a pop-up that indicates that the product requires a physician (or another provider) code, recommendation or prescription and/or initiates the proper telemedicine visit.

Such links and notices may be provided anywhere within the marketplace to prompt a perusing customer to get a consultation to determine if the particular product 304 is right for them and/or to give the healthcare provider the opportunity to close the sale and/or to upsell.

Once redirected to the healthcare provider's curated products platform 300 interface, (i.e., a physician-specific mini-store) the patient may pay for (or not, depending on the patient's benefits etc.) the visit and proceed to obtain a telemedicine consultation with the healthcare provider (e.g., a physician) in conjunction with the assistance of the pharmacist or their staff member.

In various embodiments, when a patient selects “MTM visit” the order form may be presented to capture requisite or useful data for the pharmacist to conduct the MTM visit. The curated products platform 300 can be integrated with drug, food and/or vitamin/supplement interaction monitoring software. The interface may help the pharmacist conduct an efficient and thorough MTM visit.

The telemedicine system may also provide an interface with medical supply companies, such as durable medical supply (DME) companies, diabetic supply companies, continuous positive airway pressure (CPAP) supply companies, orthopedic supply companies, and/or other medical supply companies.

The telemedicine system may provide discounts on diabetes supplies, CPAP supplies, DME, and orthopedic supplies. Similarly, the telemedicine system may provide discounts on prescriptions drugs. In some embodiments, a telemedicine visit may be preceded by a direct recommendation for services or products on one dashboard and/or as the direct result of a telemedicine consultation. Thus, the telemedicine system may drive demand and/or increase awareness of patients for particular products or services.

In some embodiments, the telemedicine system may implement a bidding process to the DME or other supply companies to be presented to a provider that selects one of the buttons for the diabetes supplies, CPAP supplies, DME, and orthopedic supplies. The bidding process may be used to provide the best price to the purchasing provider and/or to maximize the percentage collected by the telemedicine system.

Products 304 may be available via a combination or integrated e-commerce and curated products platform 300. In various embodiments, the selection of the particular product 304 may result in an automatic or offered consultation that is optional or mandatory for the selected product 304. An online clinic supported by the telemedicine system may be integrated into the healthcare provider's website. The website may allow for the selection of the healthcare provider via a drop-down menu.

In some embodiments, a patient may select the type of telemedicine visit in which they are interested. Options may include e-visits, in-office visits, and/or house calls. For each given type of appointment type, the drop-down menu of available healthcare providers may change to reflect those healthcare providers who offer the selected type of visit. In some embodiments, a patient may shop online for various classifications, categories, types, or classes of telemedicine visitations. Pricing may reflect cash-payment pricing, insurance pricing, and/or affiliation discount pricing based on a login status, coupon code, or healthcare provider selected discount.

A patient may shop for and/or schedule an in-office visit. In some embodiments, a healthcare practitioner may offer a consultation and/or product packages at reduced or bulk pricing. As an example, a service provider may charge a periodic (weekly, monthly, yearly, multi-year) fee to the healthcare provider based on the number and types of products 304 selected for inclusion on the practitioner-specific curated products platform 300. For instance, pricing models may be created for each product 304 and/or for packages of products 304. In some embodiments, a flat pricing model may be implemented in which the healthcare provider pays the service provider a flat rate (one-time or subscription-based) to create the practitioner-specific curated products platform 300 with any number or type of products 304 from the product library. In other embodiments, the pricing may be a la carte based on the specific products 304 selected. In yet other embodiments, the pricing may be based on the actual usage of each of the selected products 304 included in the practitioner-specific curated products platform 300.

In some embodiments, the healthcare provider may provide an assessment and plan to a patient that includes a click-to-order or click-to-buy link for imaging, studies, prescriptions, products, and/or services. Thus, the curated products platform 300 allows for the integration of physician-recommended-only products, lab studies, and pharmaceutical drugs that are offered for “sale” to the patient on the physician's website and which are then integrated into a mandatory online consultation.

In some embodiments, an e-commerce integrated offering of the curated products platform 300 is offered, according to various embodiments. When the healthcare provider recommends or approves the order request to buy a pharmaceutical drug, imaging study, or lab test, the healthcare provider may select the pharmaceutical, lab test, or imaging study and then send a click-to-buy button or link to the patient for purchase.

In some embodiments, the discussion module includes a messaging tab of a user interface for initiating a SecureMessageRx session using the algorithms and workflows described herein for prescription management. In various embodiments, a messaging tab in an application (e.g., on a desktop, tablet, mobile phone, or another personal electronic device) allows the healthcare provider to select “SecureMessageRx” from a menu. In various embodiments, only healthcare providers with a DEA, NPI and/or active state license number will be allowed to send a SecureMessageRx.

Similar limitations and verifications may be provided for other jurisdictions based on local laws. In some embodiments, all staff members or some staff members that have permissions granted by authorized individuals (e.g., the providers) may also be allowed to prescribe on behalf of their healthcare providers. In some embodiments, an administrator of the telemedicine system may periodically request or require providers to update their information to demonstrate that they are authorized to give prescriptions to patients.

In one example, a patient may have three products 304 selected for adding to a card, including a lotion, a cream, and a dark circle remover product. Each product 304 can have a rebate coupon associated with it. Each product 304 may have a regular coupon associated with it and/or qualify for an auto-ship discount (e.g., monthly or 90-day auto-refill supplies). The auto-ship discount may be cumulative for each product in aggregate, but each product may have an auto-ship discount.

Patients can choose “pick up,” “delivery,” or “ship” (assuming that these respective modalities are offered by the pharmacy). Patients can delete or add the number of products 304 that they want. Patients can also access the store's e-commerce products 304 and add more things to their cart as desired.

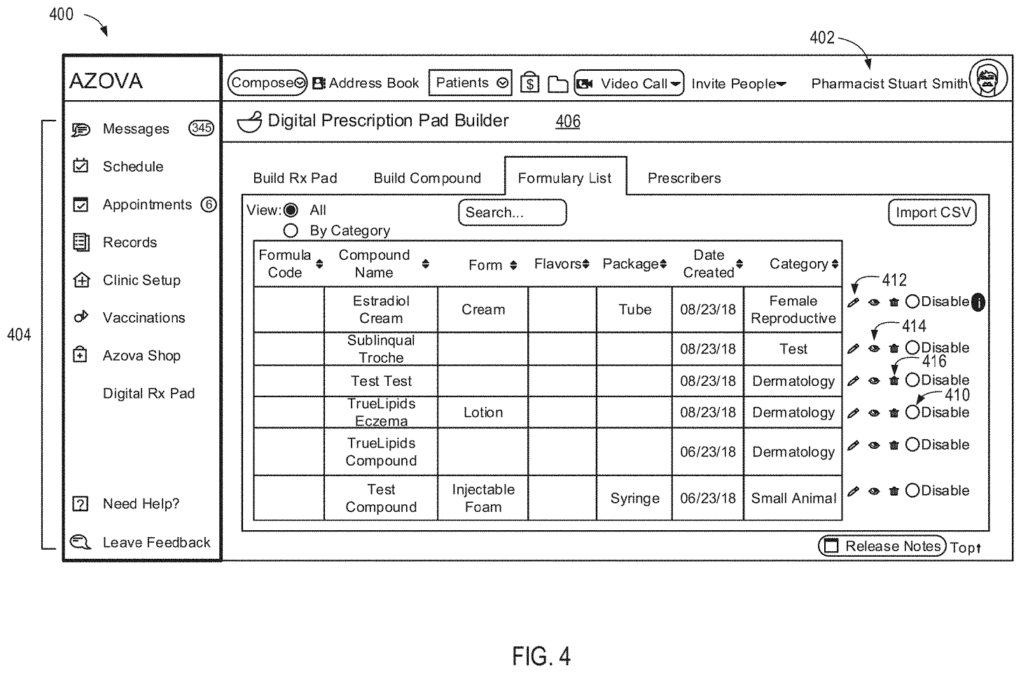

FIG. 4 illustrates a screenshot of one possible embodiment of a GUI of a healthcare provider 402 utilizing a curated products platform 400 to select a customized product 404. The curated products platform 400 may include a healthcare provider 402, the product 404, and an order form 406. The customized product 404 is developed by populating the order form 406. In some embodiments, the order form 406 may be populated by selecting from a variety of options. In some embodiments, the order form 406 may be populated by providing a product description. The product description may include a picture, a PDF, a handwritten note, or another related form.

In some embodiments, the healthcare provider 402 may utilize the curated products platform 400 to populate the order form 406. In such embodiments, the healthcare provider 402 may select from a drop-down menu, clicking bubbles or GUI. The healthcare provider 402 may select from a variety of options to make the customized product 404. For instance, the healthcare provider 402 may create a compound prescription. To create the compound prescription, the healthcare provider 402 may select from a variety of options available. In the illustrated example the healthcare provider's 402 curated products platform 400 displays the order form 406 in the form of a digital prescription pad builder. The digital prescription pad builder provides four tabs here. Provided tabs include a Build Rx Pad tab, a Build Compound tab, a Formulary List tab, and a Prescribers tab.

The formulary list tab is open displaying the compounds selected by the healthcare provider 402. Here, the compounds are displayed in the form of a list, allowing the healthcare provider 402 to utilize the curated products platform 400 to view the selected compounds. The healthcare provider 402 may also view data associated with the selected compounds, edit one or more of the selected compounds, delete one or more of the selected compounds, and/or disable one or more of the selected compounds. In some embodiments, more options may be available. In some embodiments, fewer options may be available. The healthcare provider 402 may utilize the curated products platform 400 to select options for a compound for a library of compounds. The healthcare provider 402 may utilize the curated products platform 400 to build a customized compound.

In this embodiment, the formulary list tab of the order form 406 is opened, displaying a list of compounds in the formula. In this embodiment, data includes NDC, Compound Name, Strengths Offered, Form, Package, Date Created, and Category. Additionally, in this embodiment, the formula list tab includes an edit button 412, a preview button 414, a trash button 416, and a disable button 418. The edit button 412 allows the healthcare provider 402 to edit the associated compound. The preview button 414 may enable a displayed preview of the associated compound. The trash button 416 may remove the associated compound from the formula. The disable button 418 may prevent the healthcare providers from viewing and/or selecting the associated compound.

In some embodiments, the healthcare provider 402, (e.g., a pharmacist) may build an electronic subscription pad. The pharmacist may upload APIs, flavors, packages, etc. and create a compound. The pharmacist may assign categories to the compound and then make those categories viewable by only a particular provider, provider group, or provider type. Thus, unique combinations of compounds and base compounds may be made viewable to different categories and types of providers or groups of providers.

In some embodiments, the healthcare provider 402 may select a pharmacy and enter a patient for whom the prescription will be sent. In addition to the healthcare provider 402 sending a traditional prescription, the healthcare provider 402 may select the digital prescription pad or (Digital RxPad) and can choose a category and then choose a compound.

In some embodiments, the order form 406 may be prepopulated with personal information about the patient (e.g., name, age, identification information, social security information, medical record identification numbers, and/or other patient demographic information), information regarding the healthcare professional (e.g., personal, entity, and/or other identification information), insurance information, proof of authorization to access medical records, and/or other information for accessing, requesting, and/or transferring medical records.

Products 404 may be configured and sold through a custom online store that is integrated with the online clinic and/or telemedicine consultations. Coupons can be created for any visit, any provider, or an entire group. The traditional process may include the healthcare provider 402 recommending a treatment or product 404. The patient then leaves and goes to another, unrelated entity (e.g., a pharmacy) to purchase some variation of the product 404 or treatment with potential for some deviation from the original recommendation. The presently described combination may allow for increased profits to the original healthcare practitioner and may also be instrumental in improving patient outcomes by improving compliance.

Laboratory ordering, imaging, and prescriptions may all be integrated and connected. Lab tests, prescription drugs, and imaging studies can be offered for sale on the health professional's online store or can be ordered by the health professional as the result of a consultation purchased through the online clinic. The patient can pay cash or use insurance if that option is offered by the health professional. In various embodiments, patients can select products 404 and add them to their online shopping cart and just as easily add studies, imaging, reports, prescriptions, subscriptions, lab tests, pharmaceutical products, or other items to an online cart for checkout. Some of these products 404 may already be internally associated with prescriptions based on prior consultations, and others may be automatically configured to generate a consultation request to obtain the necessary prescription.

As an example, a consultation may result in a prescription for a face cream with any of three active ingredients, each determined to be adequate alternatives by the healthcare practitioner. The prescription may be unfinished while the patient shops the variously available face creams in an online store after the consultation is over. Upon selection of a face cream with one of the three active ingredients by the patient, the unfinished prescription is automatically finished, allowing the patient to purchase the product 404. The purchase of the product 404 forecloses future purchases of more of the same product 404 or the other products 404 absent an additional prescription and/or consultation.

In various embodiments, the addition of products 404 can include lab tests, pharmaceutical products, or imaging studies and may result in a request for the necessary prescription or consultation from the appropriate healthcare provider 402. The healthcare provider 402 may have the authority to approve, cancel, or change the order for the patient and send any recommendations to the patient.

Thus, the curated products platform 400 allows for the integration of physician-recommended-only products, lab studies, and pharmaceutical drugs that are offered for “sale” to the patient on the physician's website and which are then integrated into a mandatory online consultation.

When the healthcare provider 402 recommends or approves a request to buy a pharmaceutical drug, imaging study, or lab test, the healthcare provider 402 may select the pharmaceutical, lab test, or imaging study and then send a click-to-buy button or link to the patient for purchase.

In various embodiments, vendors can set themselves up on the back end and abstract the curated products platform 400 from the end users. Vendors can offer direct to consumer sales and direct to wholesale ordering and/or can offer their products 404 only to affiliated groups. Vendors may select whether they want their products 404 on the open network. Vendor products 404 may be listed at various pricing and availability depending on affiliation status of the healthcare provider 402.

Vendors might sell directly to patients, healthcare practitioners, healthcare organizations, insurers, and/or other service providers. Products 404 may include, but are not limited to: nutritional supplements, personal care, food, office supplies, medical supplies or equipment, maintenance offerings, dental equipment, veterinary supplies or equipment, software, laboratory studies, imaging studies, a telemedicine visit, an appointment, and the like.

The same or different products 404 may be sold direct to consumers by a vendor. Products 404 may be categorized, filterable, and/or searchable. Such offerings may include items in personal care, over-the-counter items, and direct sale products (multi-level-marketing).

The systems and methods described herein can additionally or alternatively (e.g., as a stand-alone system) provide vaccine management and associated process, methods, and systems. In various embodiments, the patient or prospective patient may schedule online visits, some of which include various vaccine options and visit types.

Thus, a wide variety of healthcare management systems may include an online vaccination management system (VMS) for scheduling vaccination visits, offer in office/pharmacy patient registration for vaccines, and/or offer other vaccine related services such as record access. It may also provide a price-transparent way of selling vaccines and associated services. The telemedicine system may include inventory management to manage the stock of vaccines and reorder them as necessary. It may also limit appointment scheduling to include those appointments for which the necessary or recommended vaccines are in stock. The vaccine management system may also allow for improved care coordination among healthcare professionals.

In various embodiments, the VMS may determine an eligibility for vaccines and verify that pre-requisite vaccines and or studies have been administered. Identification of patients may be performed via usernames and other login credentials. In some embodiments, verification of identity may be conducted using credit services, third-party verification, and/or using a sovereign identity (e.g., a blockchain-based identity).

The VMS allows the healthcare provider 402 to offer online booking for in-office vaccinations of any type. When the patient signs up for a vaccination visit or for any other visit with the healthcare provider 402, the system automatically prompts the patient to create and then share an online vaccination history.

The patient can build and validate his/her online vaccine record and then share it with his school or university with the click of a button. Healthcare professionals, pharmacists and health departments can manage all of their vaccine patients from one dashboard. The VMS may allow for onsite vaccine and healthcare clinics or event-type scheduling for any number of employer groups or companies. Each company may receive a unique online clinic where employees may schedule their vaccinations or other health screenings/clinics on site without any waiting.

In various embodiments, patients and/or providers may track vaccines, utilizations, supplies, future scheduled visits, demand from prior years, etc. Such data may be useful for scheduling and/or precise inventory management decisions.

Each vaccine type may be tied to a NDC (national drug code), its wholesale and/or retail price, insurance coverage, and/or other information. A patient can choose which vaccines are wanted and the telemedicine system can add up the cost of each vaccine and/or run eligibility checks to determine if the patient's insurance will cover the vaccine and, if so, how much will be covered. The system, a third-party payor, the healthcare practitioner, and/or the patient may utilize this information and/or personal preferences to determine which vaccines, orders of vaccines, and even brand of vaccines to receive/provide.

Patients scheduling vaccines via the VMS may upload, share, or otherwise provide access to medical records on a one-time use basis, perpetually, or until such access is revoked. Patients may have options via a VMS interface (e.g., a website or mobile application) to determine which healthcare professional, pharmacist, school or university, laboratory, imaging center, family or friend with whom he/she would like to share his/her medical records.

When the patient selects the healthcare provider 402 and that healthcare provider 402 already has an account, electronic medical records and/or the request for a vaccine may be transmitted to that healthcare provider's 402 vaccination tab on his/her dashboard via the curated products platform 400. If that healthcare provider 402 does not have an account then the healthcare provider 402 may be contacted (e.g., via mail, email, SMS, phone, or the like) with a notification making available the vaccination records from the patient, the scheduling request, and options to subscribe, purchase, or otherwise become a member of the telemedicine system.

In some embodiments, the healthcare provider 404 can validate receipt of vaccines and the patient and/or another healthcare provider 404 can share the validation with schools, governments, or other entities as approved by local regulations and/or the patient. Once a vaccine is validated, a patient may be prohibited from editing it without losing the validation, but the patient may use the validated vaccine as proof of vaccine reception.

In various embodiments, a VMS system automatically presents all the vaccines that are currently FDA approved for each age. For example, certain vaccines in any one series are only approved to be used for dose four or five, but are not approved for dose 1, 2, or 3. The telemedicine system may allow the patient or provider to add vaccines that are approved for each particular dose and offers a dynamic recommended age as well as recommended time between vaccinations. These recommendations may be programmed to notify the patient when he/she is due or overdue for a vaccination and displays to the patient, and, optionally, with one or more healthcare providers 402 with whom the patient has opted shared information, that there are vaccines that are due or overdue and/or any vaccines that have been given in the past. This way, the VMS system can help to prevent over- and under-vaccination.

In various embodiments, the VMS system may aggregate various vaccination information, such as manufacturer, brand name, NDC code, lot number, an identity of an individual or facility that administered the vaccine, facility name, location of the vaccine, volume of the vaccine, location of injection and whether the vaccine was valid along with comments. As a specific embodiment, a baby may be injected with the MMR vaccine, but the nurse may not have attached the needle to the syringe tightly enough. Accordingly, the vaccine may not have been fully injected, and a large portion of it may have squirted all over the child's arm or leg. This vaccine is considered an invalid vaccine and must be recorded as such and must be made up in the future. A date may be scheduled for the makeup vaccine, and the proper entities may be notified to manage payments and discounts for the mistake.

One aspect of the VMS system may be to provide price transparency. Price transparency may allow the consumer to check benefits (e.g., cash discounts or insurance coverage) on the spot and elect to purchase procedures (or not) with confidence in the expected immediate and future costs.

Certain medications (vaccines) have been identified for increased efficacy based on a person's lab test results. This is generally referred to as “Precision Medicine” and is explained in an article titled “Personalized medicine: new genomics, old lessons” by Kenneth Offit, which is hereby incorporated by reference in its entirety as incorporated in U.S. Provisional Patent Application No. 62/378,590, to which this application claims priority.

Since vaccines are medications, lab test results will be utilized to better select which vaccines are most appropriate for a specific person, based upon their race, ethnicity, and genomic analysis. The Mayo Clinic reported in 2014 that the Rubella vaccine performed better on certain individuals, as described in the appendices of U.S. Provisional Patent Application No. 62/378,590.

The VMS system may utilize lab test results and other data from the EMRs of patients to identify the best doses, potential allergic reactions, recommended boosters, and the like to increased vaccine efficacy and safety. For instance, a gap-in-care analysis may be performed based on the data supplied by the patient, electronic health record data, data from a health information exchange (HIE), bloodwork test results, and/or results from genomics testing.

Embodiments may include various features, which may be embodied in machine-executable instructions executed by a general-purpose or special-purpose computer (or another electronic device). Alternatively, the features may be performed by hardware components that include specific logic for performing the steps or by a combination of hardware, software, and/or firmware. Any of the various embodiments may include various encryption and/or authentication measures to ensure the security and/or authenticity of the data.

In some embodiments, laboratory ordering, imaging, and prescriptions may all be integrated and connected. Lab tests, prescription drugs, and imaging studies may be offered for sale on the healthcare provider's 402 online store or can be ordered by the health provider 402 as the result of a consultation purchased through the online clinic. In some embodiments, the healthcare provider 402 may create and/or view an assessment, plan, and/or internal notes associated with an appointment, including a status identifier.

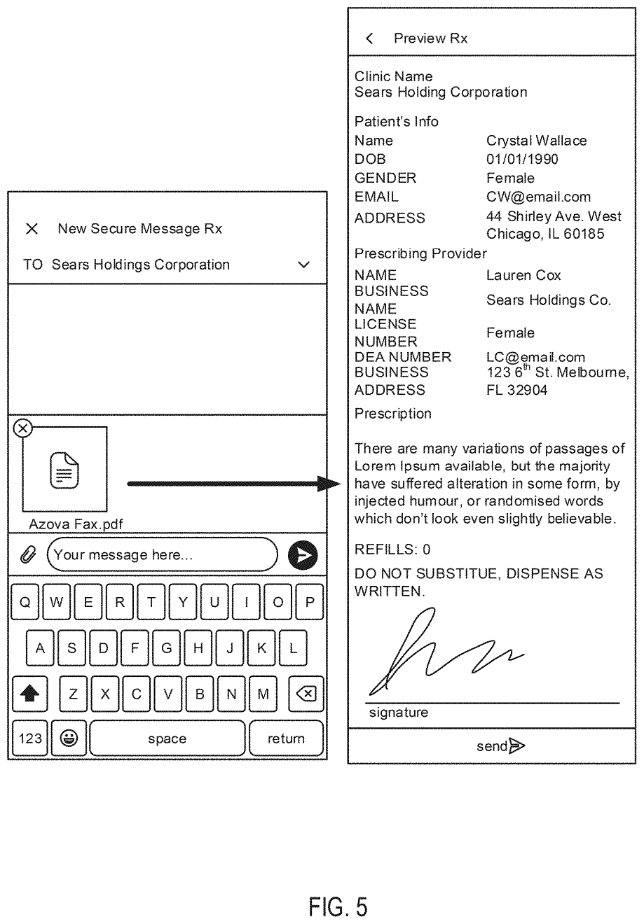

FIG. 5 illustrates a screenshot of one possible embodiment of a GUI displaying a preview of an order form. In some embodiments, a healthcare provider 502 may utilize a curated products platform 500 to populate an order form 514 via the order requisition portal. In some embodiments, the healthcare provider 502 may populate the order form 508 utilizing the input device of a computing device 510. In some embodiments, the healthcare provider 502 may utilize the computing device 510 via the curated products platform 500 to view the displayed preview of the order form 508. Before sending the order form 514, the computing device 510 may view the displayed preview of the order form 508. The healthcare provider 502 may view this displayed preview of the order form 508 to ensure the at least one correct product is being requested.

For example, a physician may populate the order form 514 with data associated with a prescription. This data may include a clinic name, patient's info (name, date of birth, gender, email, and address), prescribing provider information (name, business name, license number, DEA number, and business address), preparation information, refills, and a signature field. Reviewing this data allows the physician to ensure the correct prescription is sent to the correct patient and decreases the likelihood that an incorrected medication is delivered or that the medication is delayed.

The preview of the order form 508 may include the product name, the healthcare provider's 502 name, data relating the product, and/or any other data to assist in ordering the product. In this example, the preview of the order form 508 includes clinic name, patient's info, prescribing provider, prescriptions, refills, and a signature field. The healthcare provider 502 sending the order form 514 may utilize the displayed preview of the order form 508 before submitting the order form 514, allowing the healthcare provider 502 to ensure the correct products are being ordered. In this instance, a prescription may be sent to a clinic. The healthcare provider 502 may utilize the displayed preview of the order form 508 to ensure the correct prescription is being requested then send the order form 508 to the clinic.

In some embodiments, a discussion module 506 is utilized to display the displayed preview of the order form 508. In other embodiments, the curated products platform 500 is utilized to display the displayed preview of the order form 508. In some embodiments, the healthcare provider 502 may utilize the computing device 510 via the curated products platform 500 to display the displayed preview of the order form 508.

The order requisition portal may be utilized to populate the order form 514. In some embodiments, the order requisition portal may be utilized to allow the healthcare provider 502 to build their online clinic or online mini-shop. In some embodiments, the systems and/or methods described herein may allow for sourcing an order request. For example, a telemedicine system may receive the order request for an appointment from the healthcare provider 502. The telemedicine system may determine which staff members can assist the healthcare provider 502 and notify those staff members of the order request. The order request may then be placed in a queue until some action is taken by the staff member.

In some embodiments, an interface for selecting a pharmacy is provided. The healthcare provider 502 may enter a pharmacy name in the “To” section. As the healthcare provider 502 begins typing or finishes typing, a list of pharmacists may auto-populate based on a global address book, the provider's address book, locations near the provider, and/or locations near the patient's home address.

Alternatively, the healthcare provider 502 may search a database of pharmacies. In some embodiments, compounding pharmacies may be indicated with a “compounding pharmacy” symbol. Similarly, pharmacies that deliver may have a corresponding logo or symbol. The telemedicine system may maintain and provide a directory of clinical services available at each pharmacy, such as vaccines, medication reconciliation, diabetes management, chronic care management, transition of care management, weight management, smoking cessation, point of care testing, and/or pharmacogenomics testing.

In some embodiments, an interface for entering patient, prescription, and provider data is provided. A user may enter the patient's personal information. The user may include the healthcare provider 502, an agent of the healthcare provider 502, an invited user of the healthcare provider 502, or another user. In some embodiments, the data may auto-populate and/or be shared by the patient automatically. The healthcare provider's 502 information may autofill based on login information to include the business name, license number, DEA number, and/or business address.

Insurance information may be included in some embodiments. In some embodiments, a message may be communicated to an insurance company via the discussion module 506. In some embodiments, an insurance company may provide pre-authorization and/or specify the brand or generic types of medications that will be covered and/or at what coverage values.

In various embodiments, if a pharmacy is a compounded pharmacy and the healthcare provider 502 selects “Add Compound” then a list of formularies may be input by the compounding pharmacies.

In some embodiments, the signature field for adding a digital signature to the prescription, is provided. In some embodiments, the “signature” is a digital signature input via a touch pen, finger, etc. The digital signature may be copied from a file and look like a signature via a pen. In other embodiments, a blockchain-based authentication may be used to authenticate the provider. Various authentication, digital signature, and non-repudiation algorithms may be employed and utilized, including, but not limited to: RSA-PPS, ECDSA, DSA and elliptic curve variants of ECDSA, Rabin signature algorithm, BLS, those utilizing public-private keys, those based on blockchain identities or other public or distributed ledgers, and the like.

In one embodiment, the displayed preview of the order form 508 provides an interface for previewing the prescription before transmission via an efax service. Once the healthcare provider 502 has signed and submitted the prescription, a preview of the prescription fax and document appears. The user can select “Send” via a send button 516 to attach the prescription PDF to a secure message. The prescription PDF is sent as an efax to the pharmacy fax (traditional or digital) and/or as an attachment to a secure message in a messaging system, such as the messaging system described above.

Thus, the telemedicine system described herein may facilitate or even establish connections and interactions between healthcare providers 502, optionally through one or more translators, representatives, caregivers, and/or other intermediaries.

The staff members may come from a variety of locations. In one embodiment, the staff members may be employees of the healthcare provider 502. In another embodiment, the staff members may be employees of a third-party maintaining the system. In yet another embodiment, the staff members may be crowdsourced. For example, independent contractors may sign up to be a “staff member.” The contractors may be paid based on the appointments that they have completed. The staff members may also comprise some combination of employees and independent contractors. For instance, the telemedicine system may prioritize the use of staff members that are employees for appointments. And, if all of the employee staff members are busy, the telemedicine system may source the appointments to independent contractor staff members.

Staff members may have a profile. The profile may be configured with a set of credentials. In one embodiment, the profile may be completed by an employer. In another embodiment, the profile may be completed by the staff members themselves. For instance, when a staff member joins the telemedicine system, he/she may be prompted to insert a skill set and a schedule. The skill set may include languages spoken, specialties, or background information. Based on the skill set, the telemedicine system may automatically assign the staff members to one or more general categories (staff types). For example, if the staff member indicates he or she speaks English and Spanish, the staff member may be assigned to a Spanish interpreter staff type. Then if any appointment requires a Spanish translation, this staff member and any other in this staff type would be alerted. In one embodiment, the staff type may also indicate if the staff member is an employee or an independent contractor. In another embodiment, the staff type may also indicate the seniority of the staff member. Further, the profile may also be configured with a schedule. The staff member may indicate the hours that he or she is available for an appointment.

In some embodiments, once the staff member is registered to take appointments, the staff member may be assigned at least one handle. The handle may be a screen name, pseudonym, or unique identifier. For example, the telemedicine system may assign a name that indicates the staff type of the staff member. In situations where the staff member fits into two different staff types, two different telemedicine system names are associated with the staff member.

In one embodiment, the users of the telemedicine system may be screened by a screening module when they sign up. A user screening system may rate the user based on online activity. For example, to sign up, the user may provide the telemedicine system with access to his or her name. This may allow the user screening system to search and find any negative statements that the user has made on social media, rating sites, or other online postings. In one embodiment, the user may provide a social media account to log in. In such an embodiment, the user may agree to allow the user screening system to view any private postings. In another embodiment, the user rating may also be based on the user's credit history. In yet another embodiment, the user rating may be based on the user's legal history.

The user rating may be used to show risk associated with a user. This risk may be related to legal, financial, or publicity problems. To make a user rating, the telemedicine system may include an algorithm to derive a user score. The algorithm may be based on several variables. Each of the variables may be weighted differently depending on the significance in a specific application. For example, the variables may include how many other businesses in the same field the user has gone to in a time period. For instance, if it appears that a user is physician shopping, it would be reflected in the user score. The variables may also include a user's credit score or other credit-related information, such as bill payment history. Bill payment history may be collected from the user's interactions with the current business (when not using the system), from an affiliate, or from a credit collection agency. Further, the variables may also include the legal history of a client. For instance, if the telemedicine system is being used to provide medical consultations, any suit that the user has brought against a physician may be flagged and considered in the user score. Finally, the variables may also reflect how the user has publicly commented on other businesses. For instance, the score may reflect whether the user tends to give negative or positive feedback on online review sites.

Each variables' importance in the user score may be weighted. In one embodiment, the variables' weight is determined in part based on the field of the business. For example, the user's negative online reviews of fast food restaurants will be weighted less than the user's positive reviews of tutors when the business is an educational business. The weighting may also be done based on how recent the user's activity is.

The user score may be used to prevent the user from setting an appointment. The threshold score that determines whether the user is screened or not may be set by the owner of a business or by the individual staff members. This may be useful in fields like insurance where high-risk users are not desirable. In one embodiment, the user score may be reviewed by the healthcare provider 502 including a staff member in detail. In another embodiment, the healthcare provider 502 including the staff member may only be presented with the user score. This may help protect the healthcare provider 502 from claims over illegal screening.

In one embodiment, the user score may be continuously updated. The user's activity may be monitored after signing up with the telemedicine system. The user score may reflect the activity detected after signing up. The algorithm may then also include variables about how the appointments went, what feedback the user left, and online reviews about the telemedicine system. These activities may be significantly more weighted in the user score than the pre-signup variables. If the user score reaches the threshold score, the user may no longer be able to set an appointment. The telemedicine system may send an explanation of why the user has been screened and allow a response from the user. The response may also be taken into consideration for the user score.

An appointment may be set by the user by sending the order request. The order request may indicate what services the user is looking for and when the user is requesting the appointment. In some systems, the user may utilize a GUI of the curated products platform to select or be assigned the appropriate staff member. In such an embodiment, the user may have almost instant access to the requested product and therefore does not need to schedule the appointment. In some embodiments, the user may request a time for the service when there are an insufficient number of staff members to service an order request.

In some embodiments, the user may pay for an appointment in a variety of manners, payments methods, and time periods, including optionally via insurance or a third-party payor. In some embodiments, the telemedicine system can be set to charge a flat fee, by the minute, or a membership fee. In one embodiment where the user is charged by the amount of time, the user may set the amount of time to set a limit. In another embodiment, the user can select with which method he or she would like to pay. For example, the user may be offered a flat fee for an appointment or a certain amount per minute. Based on the user's experience, the user may decide it would be cheaper to pay the flat fee.

In some embodiments, the user may also select how it wants to interact during the appointment. For instance, the interaction may be done via live face-to-face video, telephone consultation, text, or secure messaging. The type of interaction available may be set automatically based on detected equipment. The type of interaction available may also be set based on the industry of the business.

After the telemedicine system receives the order request, the telemedicine system may place the order request in a queue. The telemedicine system may have several queues based on the services rendered. For example, in a translation service, there may be queues for each language. Each queue may be associated with a staff type. In some embodiments whenever the order request is added to a queue, the staff members assigned to the staff type associated with the queue are notified. Any of the staff members may then take the appointment request. When the appointment request has been taken, it is removed from the queue and placed into the staff members' schedule(s). Notifications that the order request has been added to the queue may be limited to only those staff members who are qualified and have an available schedule with time equal to the amount of time for which the customer wants to pay.

There may be a separate queue for employees and independent contractors. In one embodiment, the queue for employees may be limited to a certain number of users, and excess users will be placed in the queue that is available to either an employee or an independent contractor. This may allow quicker and/or more effective customer service.

In various embodiments, the systems and methods described herein may be configured to additionally or alternatively provide in-store virtual customer support. Such a system may be combined with the translation and other on-demand services and other healthcare providers 502 and sales combinations. In various embodiments, a virtual sales support system may interact with a customer to provide pertinent information about the product. The virtual sales support system may include a plurality of sales support devices. Each sales support device may be connected to a customer support network. The network may connect the sales support devices to each other and a plurality of representative devices and/or customer devices.

One reason the product might have limited sales in a retail store (e.g., a pharmacy, drugstore, etc.) is because of a lack of information and visibility. In other embodiments, the product may have limited sales because of lack of information and/or because consumers cannot differentiate between products and/or fully comprehend the advantages, uses, applications, etc. of the specific product. While many of the embodiments, described herein are related to healthcare and health supplies, this specific embodiment is universal and could be equally applied to pharmacies, hospital stores, convenience stores, grocery stores, and especially home improvement stores.

Currently, manufacturers may send human representatives to stores to present products to potential customers. For example, an air conditioner specialist may set up a table at a home improvement store, a food vendor may set up a sample station at a grocery store, or specialists may be sent to educate consumers and/or local representatives regarding specific treatments, medical devices, medications, and applications thereof. A representative may increase the sales of the product because the representative can effectively communicate information through a presentation. However, the increase of sales will be limited to the store that the representative visits, and it is cost prohibitive for the manufacturer to send a representative to every store all the time.

A virtual sales support system may allow a representative to present the product 504 and/or answer customer questions at multiple stores, on demand, and in real-time. The representative may be a staff member of the store, a sales representative from a manufacturer, and/or another employee, contractor, or volunteer person.

A sales support device may comprise one or more of a display, a microphone, a speaker, a camera, and/or a network interface. A representative may communicate with a customer via the sales support device. For example, a remote representative for a cosmetic company may demonstrate makeup products to the customer through the sales support device. The representative may appear on a display and be heard through a speaker. The customer may interact with the display and/or ask questions to the remote representative through one or more microphones. The representative can answer the questions on a remote representative device and the customer may hear the answers through the speaker.

A representative may connect to the sales support devices through a representative device. The representative device may comprise a display, a microphone, a speaker, a camera, and a network interface. The representative device may be located within a store. Alternatively, the representative device may be located remotely. In some embodiments, the representative may use a sales support device as a representative device. For example, if a representative was giving a demonstration on a toolset at a first hardware store, the representative may use a sales support device to record and/or broadcast the demonstration to other sales support devices located within the first hardware store or in a second hardware store.

The representative may be seen on multiple sales support devices at multiple stores presenting a live demonstration. For example, a representative demonstrating a smoker or grill usually can only sell to an audience at one store. With a sales support system, the representative can stream a presentation of a smoker to multiple stores and/or facilitate live questions.

In one embodiment, the sales support devices may present icons of several brands of products located in a store. More specifically, brands of products located proximate a sales support device. The brands may be different on each sales support device. For instance, the brands found on the aisles near the sales support device may be the only ones that the sales support device displays. Alternatively, the sales support device may display the brands in an order based at least partially on the location of products. For example, the sales support device may place the brands at the top that have products near the sales support device. In another embodiment, the manufacturers may pay to have their brand presented more prominently.

The telemedicine system can provide support for customers of a range of businesses. For example, the telemedicine system can be used for customer support, language interpretation, tutoring, sales presentations and promotions, legal services, banking services, medical services, training, proselyting, etc. The telemedicine system may be modified to support the various businesses. For example, a tutoring system may allow a user to communicate with video, whereas, a banking system may use secure messaging.

In one embodiment, the telemedicine system may be a unique system for each business. For example, a law firm may use a system that is uniquely configured for its clients and staff. This type of telemedicine system may be considered a closed system. The system may only source requests from the clients of a business to its own staff members. This may be advantageous for containment of sensitive information. And, because all the personnel are provided by the business, this may also help with quality control of staff members.

In another embodiment, the telemedicine system may be a common system for several businesses. The businesses may be grouped according to their field, needs, or location. In other embodiments, the businesses may self-organize and request a combined system. The combined systems may have shared consultants trained by the businesses. Alternatively, the consultants may be provided by a third-party that is maintaining the system. By combining systems, the business may receive a discounted rate for the system or the staff members.

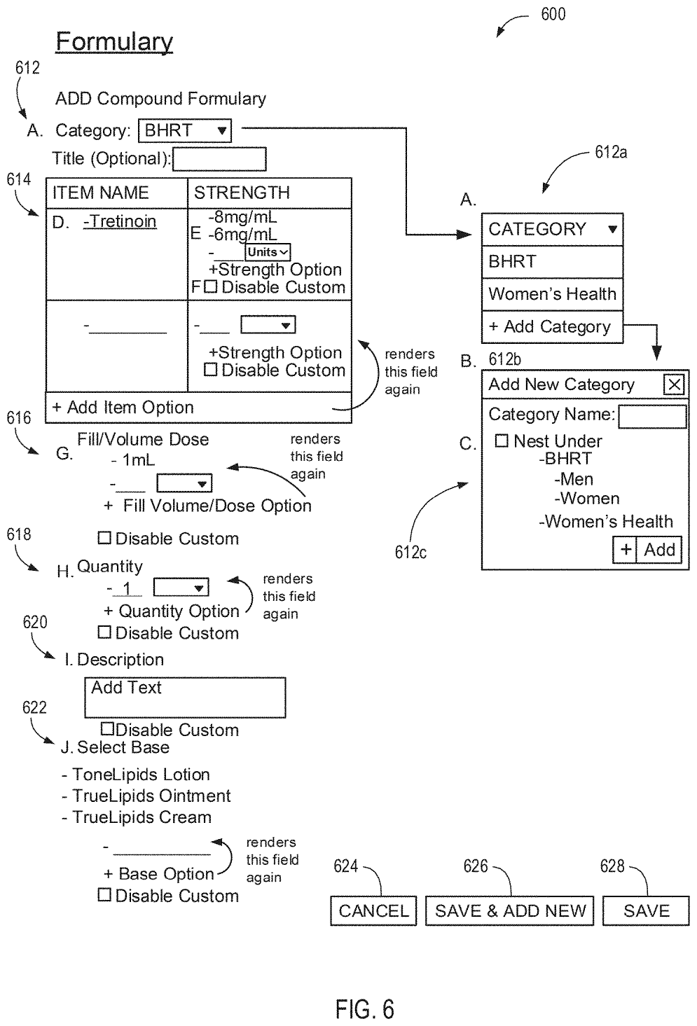

FIG. 6 illustrates one possible embodiment of a formulary builder tool interface 600. The formulary builder tool interface 600 may include selectable options for building a compound (or customized product). In some embodiments, the healthcare provider may build an electronic prescription pad. The healthcare provider may upload APIs, flavors, packages, and other related items. In some embodiments, the healthcare provider may utilize the electronic prescription pad to create a compound formula. In this embodiment, the healthcare provider may utilize the formulary builder tool interface 600 to select from a drop-down menu, such as a category drop-down menu 612. In some embodiments, particular categories may only be viewable to particular providers, provider groups, or provider types.

In some embodiments, selecting the category drop-down menu 612 provides a list of categories 612a the healthcare provider may select from. The list of categories 612a may be prepopulated and/or may include an “add category” button 612b utilized to allow the addition of a new category. In some embodiments, a list of formularies may be input by the compounding pharmacy. The healthcare provider may utilize the add category button 612b to enable a create a new category option 612c. The create a new category may be from an existing list or created by the healthcare provider. The healthcare provider may title the new category utilizing the create a new category option 612c. The create a new category option 612c may allow for selection of an existing category and nesting another category within the first selected category. For example, utilizing the create a new category option 612c the healthcare provider may select to nest men and women within BHRT. In some embodiments, the healthcare provider may select an add button 612d to add the new category to the category drop-down menu 612.

For each item added, the healthcare provider may further choose to populate a name section and a strength section field 614. The healthcare provider may name the compound and determine the desired strength of a component material (item) to be added to the compound. Additional items may be added to the compound formula by selecting an add item option. The healthcare provider may additionally populate a fill/volume dose field 616 to determine the amount of the compound to be made. The healthcare provider may additionally populate a quantity field 618. The quantity field 618 may further provide an “add quantity” option utilized to allow the healthcare provider to add additional quantities. The quantity field 618 may further provide a disable custom quantity option that may be utilized to allow the healthcare provider to disable the quantity field 618. Disabling the quantity field 618 may include preventing particular healthcare providers from viewing and/or selecting the quantity.

The formulary builder tool interface 600 may further provide a description field 620 utilized to allow the healthcare provider to describe the created compound formula. In some instances, the description field 620 may be utilized to describe the effects of the compound formula, how to use the compound formula or other information related to the compound formula. In some embodiments, the description field 620 may further include a disable custom option utilized to disable the ability to describe the compound formula. In some embodiments, the disable custom option may prevent the healthcare provider from viewing and/or selecting the description field.

The formulary builder tool interface 600 may further include a select base field 622. The select base field 622 enables the healthcare provider to add bases to the compound formula. For instance, the healthcare provider may select lotion, ointment, cream and/or another base. The select base field 622 may further provide an “add base” option utilized to allow the addition of the base. In some embodiments, the select base field 622 may further include a disable custom option utilized to disable use of the base in a new compound and/or in existing compounds. In some embodiments, disabling may include preventing particular healthcare providers from viewing and/or selecting the base.

The formulary builder tool interface 600 may further include a cancel button 624, a save & add new button 626, and a save button 628. The cancel button 624 is utilized to allow the healthcare provider to cancel an existing compound formula. In some embodiments, the data associated with the formula may be erased from the computing device, the preselected product platform, the cloud, and/or to another storage area. The save & add new button is utilized to allow the healthcare provider to save the existing compound formula and create a new compound formula. The save button 628 is utilized to allow the healthcare provider to save the existing compound formula. In some embodiments, the data associated with the formula may be saved on the computing device, the preselected product platform, the cloud, and/or to another storage area. In some embodiments, selecting the save & add new button 626 or the save button 628 may prompt the healthcare provider to send the formula or take another step within the telemedicine system.

In one example, the formulary builder tool interface 600 may only be utilized by pharmacist users (and their affiliates, such as authorized staff) who have paid to have their formularies listed on the curated products platform. The formulary builder tool interface 600 may allow pharmacists to list drug component options for the healthcare providers to select to build compounds. The pharmacist user may select the bases that are available to particular healthcare providers. Particular healthcare provider's bases may always be available, and others may be optionally included. The pharmacist user may categorize formularies for the healthcare provider to find them easily within the SecureMessageRx application.

In some embodiments, the SecureMessageRx application may be integrated as an application within the curated products platform. In other embodiments, the SecureMessageRx application may be implemented as a stand-alone platform and/or integrated as an application into a platform operated by or owned by a hospital, medical facility, provider network, pharmacy, pharmacy network, or other entity.

In some embodiments, a prescription may be pushed to both the patient's pharmacy records tab and made part of a record visible to the healthcare provider to review historical medication information. The prescription is sent as a PDF or other text- vector-, or image-based digital file. The saved record is pushed into a patient's pharmacy records tab. The record, or at least a portion thereof, may also be pushed to the provider's pharmacy record tab as well to provide historical medication data in the future.

The formulary builder tool interface 600 enables electronic prescriptions of compounded and standard prescriptions. In some embodiments, the prescription may be delivered via a discussion module eliminating the use of handwritten or facsimile transmission. The healthcare provider (i.e., a pharmacy) may upload an unlimited number of APIs, packages, flavors, doses, strength, package sizes, and other related items to create a templet for healthcare providers to utilize to create the compound formula. In some embodiments, the APIs, packages, flavors, doses, strength, package sizes, and other related items are confirmed as approved by the FDA. In some embodiments, the formulary builder tool interface 600 is utilized to allow creating prescriptions that meet the NCPDP guidelines. In some embodiments, the formulary builder tool interface 600 enables international prescribing.

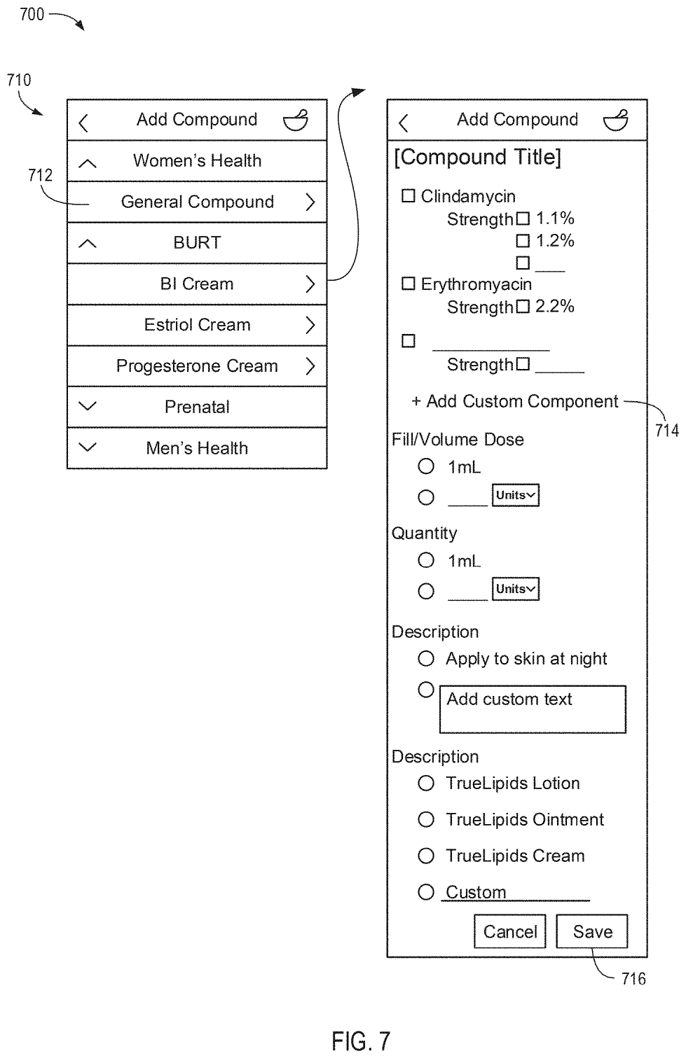

FIG. 7 illustrates one possible embodiment of an extension of the formulary builder tool interface 700 utilized to allow for the addition of a compound 712. An add compound option 710 may be provided. The add compound option 710 may be utilized to allow the healthcare provider to select the compound 712. The healthcare provider may select a category, i.e., women's health or men's health. The category may include nested categories. In this example, women's health further includes BHRT and prenatal. These categories may include previously created compounds 712. The healthcare provider may select the previously created compound 712 to populate the formulary builder tool interface. In some embodiments, the selected compound 712 may be customized. Customizing the selected compound 712 may include selecting a title, a compound, a fill/volume dose, a quantity, a description, and a base. The healthcare provider may select a compound and a compound strength. In some embodiments, the healthcare provider may select an add custom component button 714 utilized to allow the addition of the desired component.

The healthcare provider may select the desired fill/volume, quantity, description, and base. The healthcare provider may select a save button 716 utilized to populate the formula builder tool interface and/or a prescription text box with the compound 712 data.

In some embodiments, the extension of the formulary builder tool interface 700 is utilized to allow creating a prescription to be sent to a pharmacy. In these embodiments, the healthcare provider may list their formularies on a telemedicine system for healthcare providers to select or add a compound. A categorized list of formularies previously created may be displayed within or imported into the formulary builder tool interface. The prescribing healthcare provider may select the options created, or, in some cases, add a custom component. When the compound is complete, the prescribing healthcare provider selects “Save” via the save button 716 which populates the data from the extension of the formulary builder tool interface 700 into the prescription textbox of the SecureMessageRx.

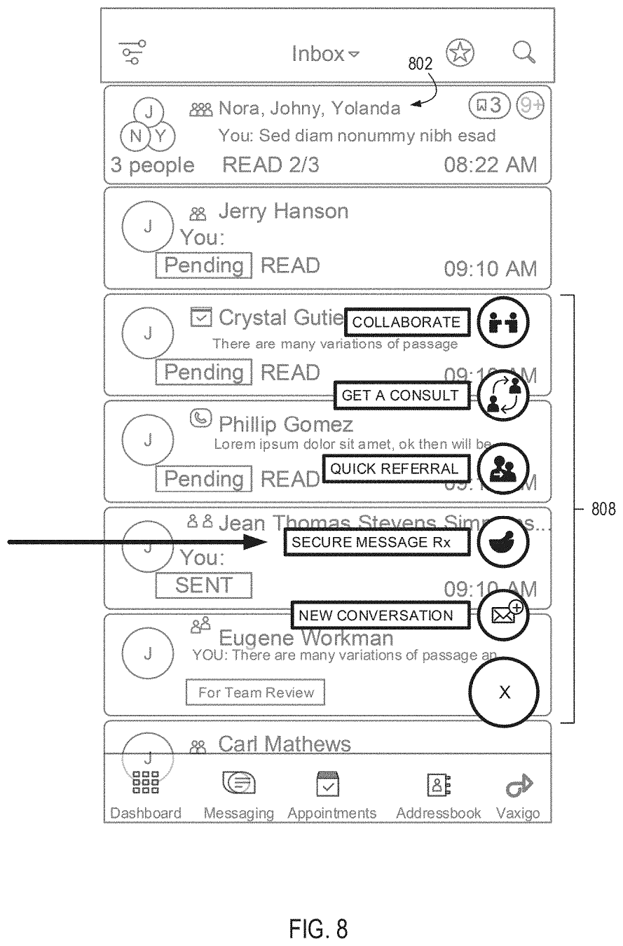

FIG. 8 illustrates a screenshot of one possible embodiment of a GUI utilized to allow communication via a discussion module 808. The discussion module 808 may include utilizing a computing device and a form of communication between one or more healthcare providers 802. Possible embodiments of the discussion module 808 may include collaborate, get a consult, quick referral, SecureMessageRx, and new conversation.

The collaborate section of the discussion module allows the one or more healthcare providers 802 to collaborate or work together. For instance, a physician and a pharmacy may collaborate to find a treatment plan for a patient. The get a consult section allows consultation between healthcare providers 802. For instance, a patient may select the get a consult section to consult a physician about an ailment. The physician may message, video conference or use another form of communication offered by the telemedicine system to assess the patient. The quick referral section may allow the healthcare provider 802 to receive a referral for a physician, pharmacy, hospital or other entity by providing information regarding their needs. The referral may be generated by a person, computing device, prepopulated list or other entity. For example, the patient may request the referral for a diabetes specialist. The quick referral section may request data such as the patient's insurer, the patient's location, and the patient's desired appointment time. Utilizing this data, the quick referral may provide the healthcare provider 802 that best matches the patient's provided information. Selecting the new conversation section may provide a new message to the healthcare provider 802. For instance, a physician may select the new conversation section then message a patient.

In some embodiments, the healthcare provider 802 may utilize the discussion module 806 to allow communication between one or more healthcare providers 802, such as between a physician and a pharmacy. The physician may message the pharmacy a prescription. In some instances, the discussion module 806 may utilize a messaging tab of the GUI to allow initiation of a SecureMessageRx session using the algorithms and workflows described herein for prescription management. In various embodiments, a messaging tab in an application (e.g., on a desktop, tablet, mobile phone, or another personal electronic device) is utilized to allow the healthcare provider to select “SecureMessageRx.” In various embodiments, only providers with a DEA, NPI and/or active state license number will be allowed to send a message via the SecureMessageRx.

In some embodiments, limitations and verifications may be provided for other jurisdictions, for instance based on local laws. In some embodiments, all members associated with the healthcare provider 802. In some embodiments, a portion of the members associated with the healthcare provider 802 are granted permissions by the healthcare provider 802 and may also be allowed to prescribe on behalf of the healthcare provider 802. In some embodiments, an administrator of the telemedicine system 802 may periodically request or require the healthcare provider 802 to update their data to demonstrate authorization to give prescriptions to patients.

While the underlying infrastructure to support secure videoconferences may be complex, the presently described systems and methods allow the healthcare provider 802 to simply select a “practitioner to physician videoconferences” product 804 from a product library. The underlying infrastructure is provided by the service provider and abstracted from the healthcare provider 802. That is, the healthcare providers 802 may be aware of the underlying infrastructure and associated complexity, but are not required to understand, manage, or otherwise concern themselves with the implementation thereof.

From the healthcare provider's 802 perspective, a selection of products 804 may be in the form of dragging and dropping the product 804 onto a webpage template, the selection of a radio button, moving an icon or text-phrase image to an “include” list, the selection of the product 804 from a drop-down menu, and/or other selection action. As used herein, the product library is not merely a collection of text, images, and/or sounds, but instead includes a library of relatively complex telemedicine products that, in many instances, are associated and/or supported by a software and/or hardware infrastructure.

For example, from a physician's perspective, selecting a video conferencing service from the product library that allows for practitioner-to-patient or practitioner-to-practitioner videoconferencing may appear as a simple video conference icon or window on a curated products platform 800. In fact, (possibly unknown to the physician), the video conferencing library product may be associated with a robust teleconferencing software solution running on remote servers that are administered, maintained, and/or paid for by the service provider.

In various embodiments, the video conference functionalities may comply with any of a wide variety of data security and/or privacy regulations, such as HIPPA. In various embodiments, the secure video conference functionalities may also allow for secure messaging (text, image, audio, document, etc.) during a video conference with one or more entities. In some embodiments, screen sharing, document sharing, image sharing, video conferences, and/or other visual communication functionalities may allow the healthcare provider 802 and/or an associated entity to draw graphics on screen (e.g., via a finger, mouse, stylus, or other input device), manipulate images, and/or otherwise provide live-time comments for viewing by both parties.

In some embodiments, face-to-face video consultations are enhanced by the ability to watch videos together (e.g., secondary videos, picture-in-picture, etc.), share documents, or otherwise collaborate. As previously discussed, the video conference services may allow for live-time commentary or markup of the videos or other shared content.

In some embodiments, advertisements may be presented to a patient while the patient is on hold waiting for a face-to-face video conference. In various embodiments, the healthcare provider 802 may select the advertisements that will be displayed. In some embodiments, the advertisements may be preceded or captioned by a notice that “Practitioner Name/Facility Name has selected the following informational videos for you to watch while you are waiting.” Accordingly, the physician can approve or even endorse the videos or advertisements displayed to the patient. The videos may be tailored to specific circumstances (diagnosis, medical history, etc.) and/or demographic information of the patient, such as gender, age, and the like. In some embodiments, the video selection may be based on a known insurance provider of the patient or a preferred vendor associated with the healthcare practitioner.

Numerous telemedicine products 804 are contemplated as being selectable products 804 within the product library. For instance, the healthcare provider 802 may select to include a store-and-forward product in the curated products platform 800. The store-and-forward product may allow a patient to upload photos, short videos, documents, and/or other information for the physician or another healthcare provider 802 to review later.

As another example, a combination of live video conferences and store-and-forward services may be selected by the healthcare provider 802. In other embodiments, the healthcare provider 802 may select to include a telephone call visit as a telemedicine service on the curated products platform 800. The telephone call visit may facilitate telephone calls between a healthcare practitioner and a patient and may create, auto-generate, and/or semi-automatically generate a medical record of the telephone call and automatically place it into long-term storage as part of the patient's personal health record.

In one embodiment, patients may be identified with specific treatments or illnesses and invited to join groups, forums, chat rooms, and/or otherwise collaborate in a secure environment with people who can relate to their current situation. Patients may be grouped based on a common illness or a common treatment plan. The data exchanged freely between these patients may be analyzed and/or otherwise data-mined for important information regarding the patients, the treatments, and/or the illnesses. The mined data may be sold to interested parties.

In various embodiments, patients may be asked to consent to receive offers associated with their medical conditions or to participate in data-gathering forums that are meant to aggregate data based upon particular diagnoses and particular treatment regimens for particular diagnoses.

The telemedicine system may also provide various services and/or functions for pharmacies, pharmacists, and/or other entities associated with MTM. For example, the pharmacists may customize the curated products platform 800 using the telemedicine system that allows them to conduct MTM visits with patients remotely.

In some embodiments, a click-to-talk or talk to us now feature may allow the patient or potential patient to immediately contact a receptionist, healthcare practitioner, or other related entity via a messaging system, a videoconferences system, an audio discussion, and/or a store-and-forward messaging (video or audio) system. Staff members of a healthcare facility may be added using a drop-down menu for specific availability scheduling for the Click-to-Talk or Talk to Us Now feature.

In some embodiments, patients may be able to share their screen with a receptionist or other staff member who can help the patient fill out paperwork (e-paper work). In some embodiments, if the staff or receptionist is not available, then a message might be received by the patient noting that the [staff type] is not currently available. The telemedicine system may then allow the patient or potential patient to sign in or sign up for a secure account to leave a HIPPA-compliant message for the healthcare facility.

In some embodiments, an intake form may be customized for particular visit types. For instance, the telemedicine system may include four different e-visit intake form types, three different in-office form types, and two different in-home visit form types. Each visit type may need a different intake form attached to it and/or require different data based on the state regulations and/or insurance expectations. A form-building tool may allow the healthcare provider 802 to customize intake and/or follow-up visit forms for particular visit types, specialties, and/or other circumstances. The healthcare provider 802 may provide their own intake forms that can be converted to digital forms and potentially added to the library of forms available to other customers.

The telemedicine system may display all of the staff members of a facility and allow the patient or prospective patient to select a desired staff member and send a secure message, schedule an e-visit, schedule an in-person visit, manage bills, view lab results, etc. Various embodiments allow patients to make appointment changes, cancel orders, etc. Automatic refunds and/or partial refunds based on the number of hours prior to the scheduled visit a patient cancels may be available.

Another feature of the telemedicine system may, in at least some embodiments, include a remote answering service (e.g., OnCallButton.com). The remote answering service can be used during or after hours to field patient calls and to contact the healthcare provider 802 in the case of an emergency.

As a specific example, a healthcare practitioner's office may record a message on its after-hours phone that states: “For after-hours consults, please go to our website at www.example.com and click on our ON CALL BUTTON. Here you can reach the On-Call physician (or another provider) via an online after-hours visit.” The message can be adapted for a particular practice and/or specific details for contacting. The message may also provide a telephone number as an alternative.

In various embodiments, the patient may call the number at which point they may be prompted to record a message for the on-call provider regarding why they need after-hours help. This message could also be recorded at the point of the initial phone call above, i.e., when the patient calls the clinic after hours in the first place. In some embodiments, the message may prompt the patient to record a message about why they need to contact the on-call after-hours provider. Once the message is recorded, or if the patient just calls the on-call number, the telemedicine system may use an IP-based telephony solution to call the on-call provider.

When the patient visits the after-hours section of the website as a result of the initial phone call (as opposed to recording a message that is routed to the on-call provider), an OnCallButton icon (or another icon that could be configured for many other uses) on the website may take the patient to a very simple telemedicine visit called an After Hours On-Line Visit (or another name as selected by the provider). The healthcare provider 802 can opt to charge for this visit or not to charge for this visit type. The visit could also be configured to load the physician/provider's full telemedicine clinic offerings if the provider has subscribed to the full the curated products platform.

In various embodiments, the patient may pay for the after-hours consultation/visit and then enter health information as prompted. For example, the patient may be asked why they need to reach the on-call provider, then enter requested medical information. Some information may be prepopulated if the patient already has an account with the telemedicine system. The patient may also be prompted to upload photos of any problem they may have. Once the patient has filled out all the required information, the patient may securely transmit the data via the telemedicine system to the healthcare provider's 802 email and cell phone (or another secure messaging system) where a message will appear that indicates that “an after-hours consultation is waiting.”

The consultation may appear inside the healthcare provider's 802 dashboard under “Consultations” or “appointments.” The physician may read the displayed data and respond electronically through the curated products platform 800 and/or can call the patient as needed.

On the healthcare provider's 802 back end, a single phone number may be used for the clinic's on-call service. This is the phone number that will be recorded on the office voicemail that the patient is supposed to call if they do not have internet access. The healthcare provider's 802 phone numbers may never be displayed. On the clinic admin dashboard, the clinic may log in to the back end to change the phone number, email and handle to those of whoever is on call. The calls, texts, and emails can be directly routed to that person after hours. The telemedicine system may have an auto-updating schedule of after-hours healthcare providers 802.

In some embodiments, the telemedicine system may include or be optionally configured to include an integrated emergency medical services (EMS) application. The EMS application may have an on button push or instant connect option that allows a healthcare practitioner, patient, and/or another user/operator to call EMS or other assigned entity or person.

In various embodiments, the telemedicine system may instantly open a streaming video interface allowing EMS to see and communicate with the person who is in the emergency situation (e.g., the patient, other person on hand such as a bystander, and/or a healthcare practitioner who was contacted first and may still be on the line).

In some embodiments, the telemedicine system may automatically and/or instantly stream video. Because the telemedicine system being used for the EMS contact is the same system that has access to the patient's EMR, the telemedicine system may provide the EMS or other emergency responder access to the patient's EMR upon request or at the same time as alerting EMS.

Similarly, the telemedicine system may provide access to emergency responders and other healthcare providers 802 in an emergency room (ER). A healthcare practitioner in the ER (or another healthcare facility) may log in to their account. A patient may log in to their account and authorize (permanently or temporarily) one or more portions of their EMR. Effectively, a patient may instantly share their entire personal health record (or a portion thereof) with any hospital, healthcare professional, or another healthcare provider 802.

As described herein with regard to other EMR sharing, the patient may share data from their personal dashboard and sending it to an interfaced EMR. The telemedicine system may create an instant message that would go to an email address where the recipient (the one who owns the email address at the ER) would receive a secure message and be instructed to log in or sign up and see the data transmitted by the telemedicine system at the instruction of the patient. Once signed up/in, the recipient will have instant access to the patient's entire medical record or the shared portion thereof.

E-visits or telemedicine consultations associated with products may utilize various technology interfaces, including face-to-face video, store and forward video/text/images, secure messaging, telephone, house calls, office visits, hospital visits, and/or in person. In some embodiments, free consultations may be offered to entice new customers or retain existing customers. In some instances, consultations that would normally cost money may be offered for free or at a discounted price if selected in the context of purchasing the product. For example, the purchase of a particular face cream or subscription to a medication may include a free telepresence consultation. Such a consultation may also be required for the purchase. For instance, a prescription medication available for purchase may be coupled to a telepresence consultation during which the healthcare provider 802 can provide the requisite prescription for the medication.

Again, while many of the embodiments and examples provided herein focus on healthcare and related fields, the preselected product platform 800 can be adapted for use with any of a wide variety of service- and product-providing industries, including medical, mental health, health and wellness, pharmacists, laboratories, imaging centers, dentists, veterinarians, lawyers, etc.

In some embodiments, the curated products platform 800 is used to configure a concierge offering for existing businesses. For example, the curated products platform 800 can be customized in a matter of minutes for integration with an existing website to provide a concierge package of products and services that can be customized for the particular business.

In some embodiments, adoption of the curated products platform 800 is encouraged by allowing patients and prospective patients to select, contact, and/or be connected with any provider, even those providers who are not affiliated with the curated products platform 800. In such embodiments, the unaffiliated provider may be encouraged to become an affiliate.

In some embodiments, an intake form or patient submission form may allow the patient, prospective patient, and/or agent of a patient to describe the reason for the visit (e-visit or otherwise). The intake process may allow the user to provide images, videos, or documents. In some embodiments, a model of a human may be shown that allows the patient to indicate where exactly the problem or issue is on the body.

Various embodiments include historical data accessible to the healthcare provider 802 to view past appointments. The historical data may include only the history relevant to the particular healthcare facility or organization, or may include all history from all health professionals, pharmacists, laboratories, or imaging centers. In such embodiments, a patient may be able to selectively hide some of the data from other practitioners.

Images and documents shared during video consultations may be edited, marked-up, and/or otherwise manipulated. In some embodiments, storage of images, documents, video, and other data may be paid for by the patient and/or the relevant healthcare organization. In some embodiments, other affiliated healthcare organizations and practitioners may be charged for accessing stored data belonging to patients and/or other organizations and/or practitioners and/or other healthcare providers 802.

In some embodiments, during an e-visit, such as a video telemedicine consultation, a patient is asked, “Would you like to record this video (or phone) consultation for future reference?” If the patient consents, a fee may apply. The fee may be charged to the patient, insurer, healthcare professional, or another person. In some embodiments, the fee may be subsidized by a third-party organization upon consent of the healthcare provider 802 to share data associated with the consultation.

The curated products platform 800 may be configured to notify the healthcare provider 802 that a particular entity is doing a study related to the type of medication, disease, product, or another aspect of a consultation and request anonymized or un-anonymized information. Incentives may be provided for those providing the desired information.

In various embodiments, the curated products platform 800 may allow for the creation and management of groups. Groups may be created that include various healthcare providers 802. A unique clinic URL can be created for each healthcare provider 802 or combination of healthcare providers 802, including staff members. As an example, a primary care physician or a mental health or wellness provider may be included in any number of other specialty clinics. These groups may include various specialists and general practitioners that are not physically near each other. Specialists may be included in multiple groups to effectively share their specialized skills between various groups, potentially minimizing the costs of care with specialists and providing an introduction of specialists into unique circles of general practitioners.

A customer may select one of the displayed icons to find out more about a brand and/or product. A customer may include the healthcare provider, an agent of the healthcare provider, or another user. The sales support device may then send an alert to a representative of that brand. The representative may then use a representative device to connect with the sales support device and interact with the customer.

For a customer needing assistance in a foreign language, the sales support device may offer on-demand translation to provide multilingual support in real time.

The representative may transfer between sales support devices. For example, a sprinkler system representative may answer a customer's question on one sales support device. Then the representative may help the customer pick out a sprinkler part by telling the customer what aisle the part is on and virtually meeting the customer thereby transferring to a sales support device near that part (i.e. virtually meeting the customer by moving between displays and associated microphones).

The customer may allow the representative to transfer to the customer's personal computing device. The customer computing device may be a portable electronic device such as a cell phone or tablet. The customer may initiate the transfer through a software application downloaded onto the customer's computing device. Alternatively, the customer may interact with the sales support device to indicate a desire to transfer the representative to a customer device. The sales support device may send a link to the customer's computing device. The customer may select the link to initiate the transfer.

The sales support devices may be used to track a customer's movements and buying habits. For example, the camera may track what items a customer picks up and what items the customer ultimately buys.

The sales support device may also include a payment module that allows the customer to pay right at the sales support device. Further, the sales support device may also present add-on options to the purchase, such as warranties and installation assistance. If the customer selects the installation assistance add-on, the customer's personal computing device may present an option to initiate an interaction with a representative of that product. For example, a home installation instruction option may appear on the customer's computing device after the customer selects the installation assistance add-on for a home theater system purchase. When the customer selects the option, a representative may appear on the customer's computing device and provide support and instructions to the customer for the home theater system.

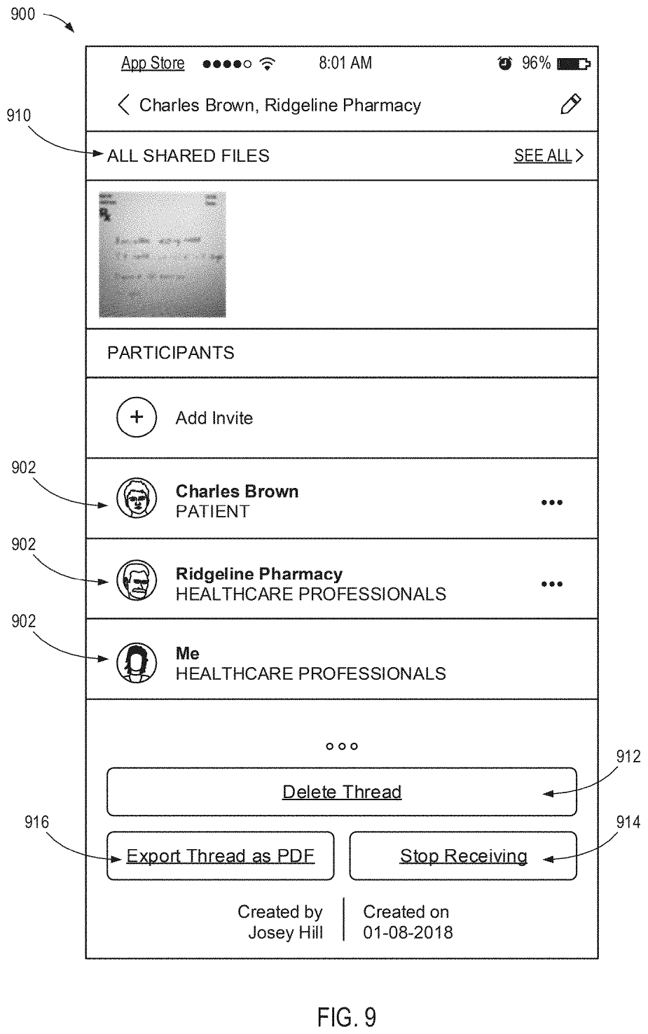

FIG. 9 illustrates a screenshot of one possible embodiment of a GUI utilized to allow communication via a discussion module 900. The discussion module 900 may include messaging between one or more healthcare providers 902. The healthcare providers 902 may utilize the messaging feature to share files. For example, a physician may send a message containing a prescription to a patient and a pharmacy. The prescription may remain in a shared file field 910 as a shared file allowing the healthcare professional 902 to review the prescription immediately or as historical data. The discussion module 900 may further include a delete thread button 912 utilized to delete the current thread. In some embodiments, the delete thread button 912 may delete the thread exclusively for the healthcare provider 902 selecting the delete thread button 912. In some embodiments, the delete thread button 912 may delete the thread from the discussion module 900 of all of the healthcare providers 902 associated with the thread. In some embodiments, the delete button 912 may delete the thread form the curated products platform. For example, a patient may be given authority to delete a thread from the curated products platform allowing the patient to retain complete control over his/her prescriptions.

In some embodiments, the discussion module 900 may include a stop receiving button 914 utilized to stop the healthcare provider 902 from receiving messages from the current thread. In some instances, the stop receiving button 914 may remove the healthcare provider from the thread. For instance, a thread may include the physician, patient, and pharmacy. The patient may no longer see the physician but the pharmacy is sending prescriptions to the patient via the thread, the physician may select the stop receiving button 914 to stop receiving messages from the thread. In other instances, the patient may want to be a part of the thread and does not want to receive notifications from the thread but wants to be able to review the shared documents/messages, the patient may select the stop receiving button 914 to stop receiving notifications from the thread.

In some embodiments, the discussion module 900 may further include an export thread button 916 utilized to export the thread into another form, i.e., PDF, JPEG, etc. In this embodiment, the export thread button 916 exports the thread to a PDF. In some instances, PDFs may increase the ease of the healthcare provider 902 to share, record, or otherwise utilize the data within the thread.

In some embodiments, the integrated secure messaging interface supports communication between the healthcare providers 902 via the discussion module 900. In this embodiment, the pharmacist, the patient, and the physician utilize SecureMessageRx (electronically securely messaged prescriptions). The physician writes a prescription, the prescription is posted back to the telemedicine system and creates a secure messaging thread between the physician, pharmacist and/or patient. Communication and collaboration are facilitated by the SecureMessageRx application, and the underlying messaging platform, such as the curated products platform, and/or another secure and compliant messaging application or format. As previously described, prescribing physicians can opt to remove themselves from the communication thread once the prescription is sent. The telemedicine system enables the pharmacy staff to create a prescription and send it to the physician and request approval or edits and electronic signature followed by returning the prescription to the pharmacy for immediate fill.

The pharmacy may create an electronic prescription pad and assign specific compounds to be displayed as an option for prescribing by the pharmacist. The pharmacy may upload products and/or categorize them. The pharmacy may make certain products viewable for recommendation by the prescriber. Such products may or may not require prescriptions.

When the prescriber selects the pharmacy, a link to prescribe a custom compound from the pharmacy's electronic prescription pad may be displayed via the discussion module 900. Alternatively or additionally, a link to a traditional prescription pad may be displayed via the discussion module 900. When the prescriber sends a prescription via SecureMessageRx via the discussion module 900, a secure messaging conversation may be initiated between the prescriber, patient, guardian, and/or pharmacist.

The telemedicine system uniquely allows for a particular compound or group of compounds or categories of compounds to be associated with a particular prescriber or group of prescribers and/or with a particular provider and/or group of providers.

When a prescriber selects a pharmacy, a link to recommend products from the pharmacy's inventory (or a select subset of the inventory—e.g., name brands only, discount products only, specials, etc.) may be displayed to the prescriber and/or sharable by the prescriber via the discussion module 900.

In some embodiments, the prescriber can only see the specific products that the pharmacy has indicated that it wants that prescriber to see. This may be based on limitations or discretionary choices made by the platform, or at the discretion of the pharmacy. That is, the pharmacy may intentionally minimize the options the prescriber has to choose from for making recommendations to a patient.

A secure messaging thread may be created via the discussion module 900 between the prescriber, pharmacist, and/or patient and enable the patient to choose to purchase the prescription, to select pick up, delivery or ship, rebate, auto-ship discount, etc.

The pharmacy may have thousands of SKUs or product numbers and hundreds of categories or subcategories of products. Any given prescriber may only have to select his/her recommendations from a small collection of products. In some embodiments, if the desired product is not saved in a provider's “favorites” or made available by the pharmacy by default, the provider may be able to add them manually and/or search for them within a complete inventory. The prescriber's “favorites” may be set up by the pharmacy, and not selected or uploaded by another healthcare provider 902.

In some instances, the discussion module 900 may include a patient medical record share portal that shows medical records of the patient with dates, provider, specialties, practices, reasons for visits, and the current number of times or people with whom the medical record has been shared. Selecting the share count may allow the patient to manage the sharing privileges relating to that particular medical record.

In various embodiments, the patient may share EMR data with healthcare providers 902 who are subscribers or members of the telemedicine system (or another service provider) and/or with healthcare providers 902 who are not members or subscribers. For example, in one embodiment, a patient may enter an email or telephone number of the physician who is not a subscriber to the system. The telemedicine system may then contact the physician using the telephone number and make them aware that EMR data has been shared. The physician may download or otherwise be provided with access to the EMR data and/or may be invited to become a permanent or temporary subscriber.

In various embodiments, patients or other users of the telemedicine system may be able to securely share EMR data with any other person (not just healthcare providers 902) by entering contact information that the telemedicine system via the discussion module 900 will use to contact the intended recipient. As a specific example, if a patient recently received an ultrasound of a baby, that ultrasound may be part of an EMR and accessible by the patient within the telemedicine system. The patient can choose to share the ultrasound with any number of people by simply entering the contact information of the intended recipients. In various embodiments, the patient may select to share the ultrasound in a secure environment (e.g., within the discussion module 900 or other portal associated with the telemedicine system) or outside of a secure environment (e.g., within the discussion module 900 via an unsecured email or MMS message).

The telemedicine system may provide a list of practitioners within a network, known to the patient, entered in the system, and associated with a particular healthcare facility; a list of current subscribers to the telemedicine system; and/or another list of healthcare providers 902. The telemedicine system may also provide a list of “current shares” and allow the patient to rescind the sharing of the particular medical record with respect to one or more of the “current shares.”

Whether through an independent personal account or through one or more of their healthcare provider's 902 practitioner-specific portals or curated products platforms, patients may have access to and control of their radiology study and associated medical records via a personal radiology study and medical record storage suite. In some embodiments, healthcare providers 902 may be charged for maintaining a database of medical records and/or radiology images/studies assessable via the discussion module 900.

The personal radiology study and medical record storage suite may allow the patient to control access to their radiology studies and associated records. In one embodiment, patients may have the ability to request their radiology or other studies (ultrasound, echocardiograms, etc.), and have them uploaded to the system or made available via a short-term or long-term portal.

There may be a one-time or subscription-based fee charged to the healthcare provider 902. Any of a wide variety of financial models might grant access to the study for an unlimited (or limited) amount of time. All or part of the studies and associated images, graphs, measurements, or the like may be accessible to the patient, and a subset of that data (or all of it) may be shareable by the patient with other healthcare practitioners, insurers, and/or other third parties.

In some embodiments, if a patient selects to share the data with the member healthcare provider 902, the member healthcare provider 902 may receive a notice that the data has been made available and access it via the discussion module 900. If the healthcare provider 902 with whom the data has been shared is not a member, the telemedicine system may contact the healthcare provider 902 (e.g., via email, phone, text, letter mail, etc.) and provide an option for one-time secure viewing of the EMR and/or invite the healthcare provider 902 to become a member for continuous access to the shared EMR. As previously described, fees may be charged to any of the parties involved for uploading, viewing, sharing, access, and/or the other features and services provided by the telemedicine system.

In some embodiments, a patient medical record share portal that shows image records with dates, provider names, reasons for the visits, and the current number of times or people with whom the image record has been shared. Selecting the share count may allow the patient to manage the sharing privileges relating to that particular image record. Again, patients may manage sharing between healthcare practitioners and non-healthcare practitioners alike.

The discussion module 900 may include a free, pay-per-use, or subscription model to charge for the secure messaging application. Any or all parties involved and/or third parties (e.g., an insurance company) may pay for usage of the secure messaging features. The sharing management features may be limited to sharing between the healthcare providers 902. Alternatively, patients may be able to share, securely or otherwise, EMR data with any person using the contact information of the intended recipients.

The secure messaging features may utilize personal data to create an account for each individual or entity (e.g., patient, healthcare facility, healthcare practitioner), such as an email address, cell phone number, or other identification. A phone application or application on a desktop, laptop, tablet device, watch, and/or other personal electronic device may allow for secure communication that is compliant with the Health Insurance Portability and Accountability Act (HIPAA), aspects of which are sometimes referred to as the Health Information Patient Privacy Act (HIPPA) (hereafter “HIPPA” is used interchangeably with HIPAA). Additionally, as used herein, anything described as complying with or associated with HIPAA or HIPPA also includes compliance and conformity to the requirements of the Health Information Technology for Economic and Clinical Health (HITECH) Act as well.

In one embodiment, the physician may receive a certain data amount (e.g., 1 Gb) from patients before their patients are billed. In other embodiments, patients are billed each time they upload an image or other media content. In some embodiments, the messaging is free to patients communicating with member-physicians but billed at a pay-per-use rate for communications with non-subscriber-practitioners.

In some embodiments, a “PhotoSafe” storage feature may be coupled with the messaging features via the discussion module that allows for photos, videos, audio recordings, images, charts, measurements, etc. to be stored in a HIPPA-compliant manner within an application on a desktop, laptop, tablet, mobile phone, or another personal electronic device. The PhotoSafe application may include a camera icon within the application that launches the device's camera and allows for the healthcare providers 902 to instantly upload a photo to the patient's EMR.

In various embodiments, the PhotoSafe application may help reduce the liability associated with photos and other protected information being lost or stolen from cell phones, unsecured messaging systems, personal or otherwise unsecured email accounts, and the like. PhotoSafe may provide for various encryption and data authenticity verification safeguards.

In various embodiments, the healthcare provider 902 or other approved entity may be able to utilize and/or manipulate photos (and/or other multimedia) during live video consultations with patients. For example, the healthcare provider 902 may conduct a live video conference and bring up a photo (or other multimedia type) and show it or portions of it to the patient. The physician may have various tools for manipulating, annotating, redacting, editing, cropping, enhancing, and/or otherwise manipulating the photo (or other multimedia type) in real-time during the consultation. The application may allow the edited/manipulated photo to be saved for subsequent recall. In some embodiments, edits may be destructive. In other embodiments, the edits may be made as non-destructive annotations to the original file. Various versions may be saved of each manipulated file as well.

As an example, a plastic surgeon or another surgeon may be able to show real-time variations to a photo or video of a patient to illustrate one or more potential surgical outcomes. The surgeon may be able to show estimates and manipulate an image as the surgeon explains a procedure or possible outcomes of a procedure.

In some embodiments, the telemedicine system may include an eConsent portal. This may be provided as part of a patient account and/or may be a product selected for inclusion in a healthcare provider's curated products platform. The eConsent portal may allow a patient to securely eSign all of their eConsents and provide audit trails showing how and when documents were eSigned. This may be implemented as a stand-alone feature and/or may comprise an integration portal with a third-party e-signing company.

In some embodiments, the healthcare providers 902 may have access through the eConsent portal to numerous (potentially more than 17,000) unique HIPPA-compliant forms. These forms may be accessible and used to import, export, request, share, make public, or otherwise control access to EMRs. In various embodiments, the healthcare provider 902 can upload and deploy unique HIPPA consent forms, office policies forms, or other forms to their patients prior to a telemedicine visit.

In various embodiments, the healthcare provider 902 may incorporate an online store into their curated products platform and/or into their existing webpage or web portal. The online store may, in some embodiments, be a “product” as described above that is selectable from the product library when the healthcare provider 902 is creating the curated products platform.

The online store itself may be customizable and allow each healthcare provider 902 to create their own mini-store of a subset of products selectable from a master list of products. The mini-store may be accessible to patients of the healthcare provider 902 to browse and shop. Alternatively, the mini-store may or may not be browse-able by patients. The healthcare provider 902 may make treatment recommendations to the patient that includes a list of treatment products that must be purchased.

The online store may also include a wholesale account link that allows physicians, suppliers, patients, administrators, and/or other entities to create wholesale accounts with any distributor company that sells products through the general online store. Such embodiments may allow companies to set up wholesale accounts quickly and seamlessly, potentially without the involvement of sales representatives. A vendor may create an interface for a wholesale account to set up a process or product and then market their products and services to any physician, including those who have previously selected to sell that vendor's specific products in their store. The vendor may “push” new products directly to a physician's store based on a pre-arranged agreement. The mini-store can also be used as a marketing portal to end purchasers and providers.

An administrator of the online store, such as the healthcare provider 902 or another manager of the online store, may be able to monitor the online store and manage, approve, review, characterize, restrict access to, and/or otherwise manage the products that are uploaded. In some embodiments, a vendor may upload products to the online store based on prior approval and/or for subsequent approval. The vendor may indicate whether the product or set of products should be made available by prescription only or by a physician or healthcare professional recommendation only.

If the product is marked as “by physician/practitioner recommendation only” or “by prescription only,” the patient may be able to select the product for purchase from the online store, but it may not be shipped or delivered until approved by the healthcare practitioner and/or a prescription is confirmed.

In some embodiments, the selection of such an item by a patient or prospective patient may result in a pop-up warning or window making it clear that the item cannot be shipped until approval is confirmed. In some embodiments, a patient may be presented with an opportunity to obtain a prescription or other practitioner approval. For instance, a pop-up window or webpage may be presented to the patient offering an in-person, remote, video conference, or other consultation for the patient to potentially obtain the necessary recommendation and/or prescription.

As a specific example, a pop-up window may state that “The following product(s) cannot be shipped to you without a prescription or health professional recommendation. Please click here to get one.” The patient may then be routed to a Prescription or Product Request page where a message is generated for a relevant or appropriate healthcare provider 902 that identifies the products that the patient has purchased and potentially other relevant patient information. In some embodiments, the patient may automatically be requested to provide health-related information that is pertinent to the requested products.

As a specific example, the healthcare provider 902 may be presented with a message that says “Your patient (or potential patient), NAME, has ordered the following items. Will you issue a recommendation (or prescription) for these products?” If the healthcare provider 902 responds in the affirmative, then the order request may immediately be sent to the vendors. Alternatively, the healthcare provider 902 may respond in the negative and the patient may be refunded and the products will not be shipped to the patient. In some embodiments, the healthcare provider 902 may recommend related or alternative products. For example, the healthcare provider 902 may approve some of the products and not others.

In some embodiments, a healthcare facility may include a mini-store of items that are accessible to healthcare practitioners associated with the healthcare facility. Such a mini-store may include supplies, clothing, medical devices, and/or other equipment commonly used by healthcare practitioners.

In such an embodiment, the healthcare facility may utilize the telemedicine system to track inventory and usage of supplies and equipment by internal healthcare practitioners. Such a system may allow healthcare practitioners to “pay” for items on an account basis that simply provides for internal monitoring. Purchases made under such a system may be shipped by the telemedicine system or simply routed for internal shipping to a supply manager of the healthcare facility.

The telemedicine system may allow for the integration of a telemedicine visit into the product description page of any product or service. For example, the mini-shop marketplace described herein may include products that are physician-dispensed only products. These products may require a physician recommendation, code, or prescription. A link to a providers' telemedicine clinic may be displayed on the product page so that patients/shoppers can select it and get the appropriate recommendation for the product (potentially via a telemedicine visit with a physician or pharmacist). The product “buy buttons” may trigger a pop-up that indicates that the product requires a physician (or another healthcare provider 902) code, recommendation or prescription and/or initiates the proper telemedicine visit.

Such links and notices may be provided anywhere within the marketplace to prompt a perusing customer to get a consultation to determine if a particular product or service is right for them and/or to give the provider the opportunity to close the sale and/or to upsell.

In various embodiments and/or in combination with any of the other embodiments described herein, the telemedicine system may allow corporations, employers, insurance companies, and/or other groups to form wellness communities. These communities can utilize healthcare providers 902 who are contracted with and/or employed by the telemedicine. Alternatively, the wellness communities can utilize their own physicians or other independent physicians. Any or all parties involved may utilize any or all of the software solutions described herein.

The telemedicine system may allow the pharmacist to configure a dashboard to allow for MTM visits and to facilitate telemedicine visits with other associated healthcare providers 902. This facilitation “visit” type allows the pharmacist to charge in exchange for taking the time to help a patient to get care with distant or remote healthcare providers 902.

In such an embodiment, a pharmacist's “Online Clinic” may include an “MTM visit,” for which the pharmacist may bill the patient's insurance, the patient, and/or the associated healthcare provider 902.

The pharmacist's online clinic may also include a “Help with an Online Visit” that directs the patient to a page that explains that the pharmacist can help them to use technology to see any healthcare provider 902 in their state who subscribes to the telemedicine system. The pharmacist may set a fee for this type of help/visit/facilitation. Once the patient has paid for this visit (automatic billing may bill the patient later), the patient may then select the healthcare provider 902 available via the telemedicine system by entering the handle of the healthcare provider 902.

Once redirected to the healthcare provider's 902 curated products platform interface, the patient may pay for (or not, depending on the patient's benefits etc.) the visit and proceed to obtain a telemedicine consultation with the healthcare provider 902 (e.g., a physician) in conjunction with the assistance of the pharmacist or their staff member.

In various embodiments, when the patient selects “MTM visit,” a form may be presented to capture requisite or useful data for the pharmacist to conduct the MTM visit. The curated products platform can be integrated with drug, food and/or vitamin/supplement interaction monitoring software. The interface may help the pharmacist conduct an efficient and thorough MTM visit.

In one embodiment, the discussion module 900 is utilized to see, review, and/or schedule a visitation with any healthcare provider 902 on behalf of themselves or another. In various embodiments, visits of any type can also be customized based on a referral from another healthcare provider 902. In some embodiments, in-office appointments and procedures can be offered and scheduled by a patient without generating a phone call.

Offerings may include products, some of which may be coupled with consultations. Memberships and packages may also be offered. Thus, the healthcare provider 902 can offer a combination of products and package them together. Such combinations may be offered at discounts and may include one-time purchases, monthly subscriptions, and/or another periodic recurrence.

In some embodiments, the discussion module 900 may provide an online intake form for the patient or potential patient. In some embodiments, an intake form or patient submission form may allow the patient, prospective patient, and/or agent of the patient to describe the reason for their visit (e-visit or otherwise). The intake process may allow the user to provide images, videos, or documents. In some embodiments, a model of a human may be shown that allows the patient to indicate where exactly the problem or issue is on the body.

In some embodiments, the discussion module 900 provides for displaying historical consultations and visits. Historical data may be made accessible to the healthcare provider 902 to view past appointments. The historical data may include only the history relevant to the particular healthcare facility or organization or may include all history from all health professionals, pharmacists, laboratories, or imaging centers. In such embodiments, the patient may be able to selectively hide some of the data from other practitioners.

In some embodiments, the discussion module 900 provides for an automatic appointment reminder generated via the curated products platform that can be customized. In some embodiments, the discussion module 900 may provide for a video conference picture-in-picture that can be used as part of a telemedicine consultation. In some embodiments, the video conference picture-in-picture may be utilized as a portion of a telemedicine consultation. In some embodiments, during an e-visit, such as a video telemedicine consultation, a patient is asked, “Would you like to record this video (or phone) consultation for future reference?” If the patient consents, a fee may apply.

In some embodiments, the discussion module 900 may provide a consultation or appointment status and notes. A status of a consultation or visit may be accessible to the healthcare provider 902 to allow for easy tracking of next-steps or outstanding tasks. In some embodiments, a change in the status of a consultation may trigger a notification to relevant parties. For instance, each update may be sent to a patient to inform the patient as to what is being done for them (e.g., “Your order has been sent to the lab” or “Prescription sent to the pharmacy”).

Once the pharmacist has received the SecureMessageRX, the pharmacist can clarify insurance and payment information with patient and insurance company, coordinate delivery and/or pickup time and location, collect prepayment before filling or compounding the prescription, send a secure message to the physician for clarification or to request changes, and/or the like. In various embodiments, prescription workflows may be provided via the application with status indicators for various phases of the process, including, but not limited to: prescription receipt, insurance info received, payment info received, queued prescriptions for compounding or filling, prescription filled, ready for pickup, delivery started, delivered, picked up, etc. Such indicators may be provided to staff, nurses, pharmacists, providers, etc., depending on applicability.

In some embodiments, the healthcare provider 902 of any of a wide variety of professional service types may register for the curated products platform.

Customization of the telemedicine system may be used by any of a wide variety of businesses to conduct instant live, face-to-face video or store and forward “consultations” for prospective and established clients. Industries for which the telemedicine system can be adapted include, but are not limited to, law, sales, insurance sales and brokerage, architectural consultation, education, retail stores, consumer products and more.

The telemedicine system can be adapted for any of these industries to do “Online Specials” in conjunction with an in-person face-to-face video consultation or a store and forward or other online or digital consultation type (including telephone). The On-Call Button can also serve as an answering service for these businesses. The use of the telemedicine model can also be adapted for one or more of these industries. All the features of the telemedicine system can be configured specifically for each industry.

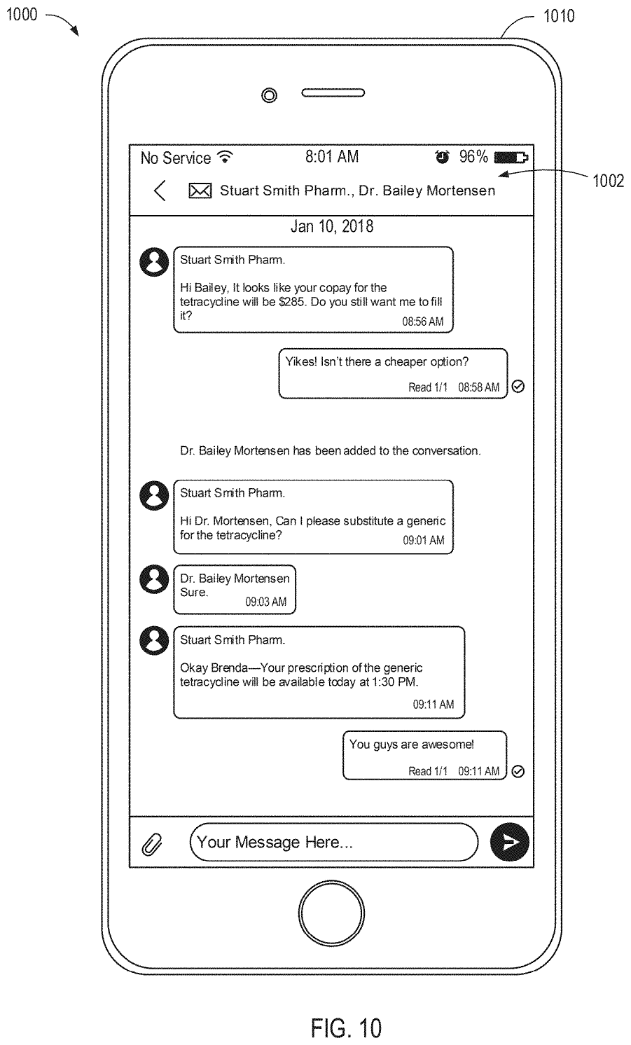

FIG. 10 illustrates a screenshot of one possible embodiment of a GUI utilized to allow communication via a discussion module 1000. In some embodiments, the discussion module 1000 may include a computing device 1010. In this embodiment, the computing device 1010 is a cellar device. In other embodiments, the computing device 1010 may include any computing device. In some embodiments, the computing device may further include a network and a server. The network may facilitate communication between the server(s) and the client devices. The computing device 1010 is utilized to allow communication between one or more healthcare providers 1002 via the discussion module 1000. In this embodiment, the healthcare providers 1002 utilize the discussion module 1000 via the computing device 1010 to message each other. In this instance, the physician, pharmacist, and patient are messaging to ensure the patient receives the correct medication. The discussion module 1000 enables the patient to request a cheaper version of the prescribed medication. Utilizing the discussion module 1000 of the curated products platform decreases the patient's wait time to receive the correct cheaper version of his/her medication.

Many of the embodiments described herein may be implemented and/or provided in the form of a computer program product, such as a non-transitory machine-readable medium having stored thereon instructions that may be used to program a computer (or another electronic device such as a controller, processor, or microprocessor) to perform processes and operations described herein. The machine-readable medium may include, but is not limited to, hard drives, floppy diskettes, optical disks, CD-ROMs, DVD-ROMs, ROMs, RAMs, EPROMs, EEPROMs, magnetic or optical cards, solid-state memory devices, or other types of media/machine-readable medium suitable for storing electronic instructions.

The various functional components of the described systems and methods may be modeled as a functional block diagram that includes one or more remote terminals, networks, servers, data exchanges, and software/hardware/firmware modules configured to implement the various functions, features, methods, and concepts described herein. In many instances, each application, embodiment, variation, option, service, and/or another component of the systems and methods described herein may be implemented as a module of a larger system. Each module may be implemented as hardware, software, and/or firmware, as would be understood by one of skill in the art for the particular functionality, and may be part of a larger physical system that may include computer-readable instructions, processors, servers, endpoint computers, and/or the like.

Disclosed herein are embodiments of systems, methods, apparatus, circuits, and/or interfaces. As stated above, the embodiments disclosed herein may be embodied as executable instructions stored on a non-transitory machine-readable storage medium. The instructions may comprise computer program code that, when executed and/or interpreted by a computing device, causes the computing device to implement the processing steps and/or operations disclosed herein. The embodiments disclosed herein may be implemented and/or embodied as a driver, a library, an interface, an application programming interface (API), firmware, Field Programmable Gate Array (FPGA) configuration data, and/or the like. Accordingly, portions of the embodiments disclosed herein may be accessed by and/or included within particular modules, processes, and/or services (e.g., incorporated within a kernel layer of an operating system, within application frameworks and/or libraries, within device drivers, in user-space applications and/or libraries, and/or the like). Alternatively, or in addition, the embodiments disclosed herein may be implemented as particular machine components, which may include, but are not limited to: circuits, processing components, special-purpose processors, general-purpose processors, interface components, hardware controller(s), programmable hardware, programmable logic elements, FPGAs, Application Specific Integrated Circuits (ASICs), and/or the like.

The VMS system provides a technical solution to a problem originating in the computer implementation of vaccination management. The functionalities of the various modules provide significantly more functionality than the mere computerization of standard vaccination management that is performed manually (e.g., via pen and paper). Moreover, the presently described embodiments to not tie up the automation of vaccination management—rather they provide a unique and specialized vaccination management system that provides a specific solution in specific instances and to specific problems, many of which did not exist prior to the computerization of health records and vaccine management in particular.

Software offerings available may include software (e.g., computer programs or applications for portable electronics) for practitioners, organizations, or individuals (e.g., patients). The described features, operations, or characteristics may be combined in any suitable manner in one or more embodiments. The order of the steps or actions of the methods described in connection with the embodiments disclosed may be varied. Thus, any order in the drawings or Detailed Description is for illustrative purposes only and is not meant to imply a required order.

The embodiments disclosed herein improve the operation of the computing device 1010 by, inter alia, enabling coordination between separate, standalone applications operating on the computing device. The embodiments disclosed herein improve the operation of networked computing devices by, inter alia, enabling coordination between separate, standalone applications operating on disparate computing devices. Accordingly, the embodiments disclosed herein may provide additional functionality that does not exist in the general-purpose computing device 1010 and/or may improve the operation of the computing device 1010 by coordinating the operation of general-purpose applications that do not include coordination-specific functionality. Accordingly, the embodiments disclosed herein may improve the operation of the particular applications operating on the computing device and/or improve the operation of particular applications normally operated on disparate and distinct computing devices 1010.

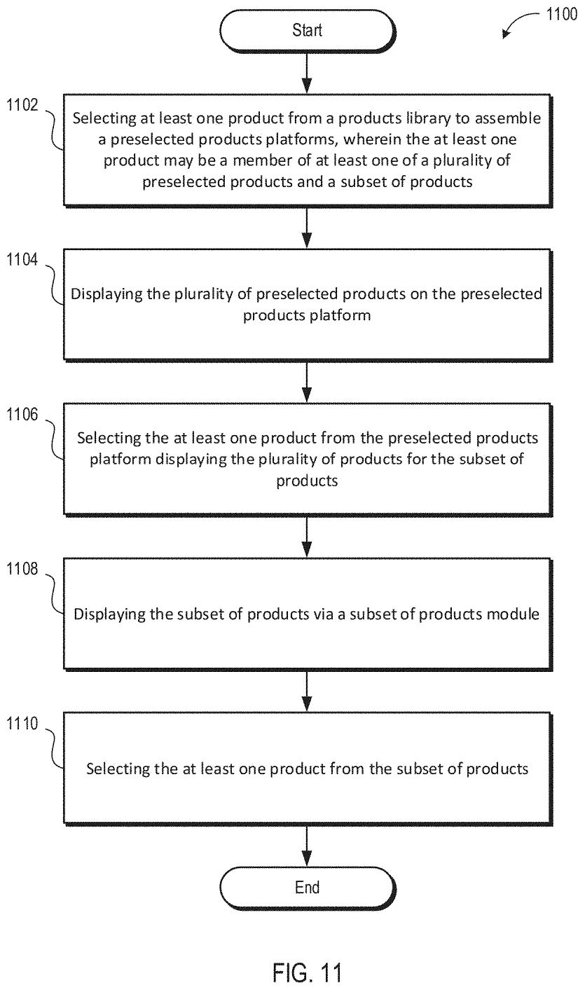

FIG. 11 illustrates a flowchart of a method 1100 for displaying preselected products consistent with embodiments of the present disclosure. In some embodiments, a library of products is populated. In some embodiments, the product library is prepopulated. At 1102, a healthcare provider selects products from the product library to assemble a curated products platform. In some instances, this may be a pharmacy creating an online store. At 1104, a plurality of products is displayed on the curated products platform to the healthcare provider. In some instances, this may be the online store created by the pharmacy being displayed to physicians. At 1106, the healthcare provider selects products from the plurality of products displayed on the curated products platform for a subset of products. At 1108, the subset of products is displayed on the curated products platform to the healthcare provider. In some instances, this may be prescriptions and/or related items sent to a patient. At 1110, the healthcare provider may select products from the subset of products. One or more of these products may be purchased by the healthcare provider. For instance, a patient may receive a subset of products from his/her physician then select one or more of the products to purchase. The subset of products ensures the patient is receiving the proper treatment and decreases time spent searching for the proper products. Additionally, in some embodiments, the curated products platform decreases the amount of time spent searching for products and/or increase profits. For instance, a plastic surgeon may search a curated products platform created by a pharmacy specifically for the plastic surgeon, the plastic surgeon may decrease time searching for products, may discover unknown and/or more effective products, and/or increase profit searching from products purchased by the patients.

The telemedicine system is an internationalized platform. The systems and methods herein may be used internationally and by customers of various languages and cultures. Thus, the curated products platform can be used and customized for any country and/or language in the world. The curated products platform may allow a patient to communicate/have a telemedicine consultation with any provider anywhere in the world. Customization of the telemedicine system may be used by any of a wide variety of businesses to conduct instant live, face-to-face video or store and forward “consultations” for prospective and established clients. Industries for which the telemedicine system can be adapted include, but are not limited to, law, sales, insurance sales and brokerage, architectural consultation, education, retail stores, consumer products and more. The use of the telemedicine model can also be adapted for one or more of these industries. All the features of the telemedicine system can be configured specifically for each industry.

The telemedicine system can be adapted for any of these industries to do “Online Specials” in conjunction with an in-person face-to-face video consultation or a store and forward or other online or digital consultation type (including telephone). The On-Call Button can also serve as an answering service for these businesses. The use of the telemedicine shop model can also be adapted for one or more of these industries. All the features of the telemedicine system can be configured specifically for each industry.

Many changes may be made to the details of the above-described embodiments without departing from the underlying principles of the present disclosure. Moreover, all combinations and permutations of each of the embodiments and functions described herein are contemplated and may be useful in a particular application.

The systems and methods described, including sub-portions thereof, specific features, algorithms, and the like may be implemented as modules in hardware, firmware, and/or software. Computer processors, servers, mobile applications, authentication techniques, encryption techniques, memory, storage, data backup technology, and a wide variety of other hardware may be custom tailored and/or utilized to enable the above-described systems, methods, algorithms, and processes. The various system models described and discussed herein would be appreciated by one of reasonable skill in the art.

Additional understanding of the embodiments of this disclosure may be gained by reference to the drawings. Numerous specific details are provided for a thorough understanding of the embodiments described herein. However, those of skill in the art will recognize that one or more of the specific details may be omitted, or other methods, components, or materials may be used. In some cases, operations are not shown or described in detail. For example, well-known features and functions normally employed in other fields of use that are incorporated in the presently described embodiments in new ways are only described to the extent necessary to understand the integration of the features and functions in the respective embodiments of this disclosure.

1. A system to initiate healthcare consultations in response to a request to purchase a healthcare product, comprising:
a network communication module to connect server devices to client devices, including remote client devices; and
a server to communicate with remote client devices via the network communication module, the server including:
a user interface module to provide a patient user interface (UI) to a first client device enabling a patient using the first client device to view and select a product for purchase from a plurality of healthcare products that are available for shipping to the patient that require a prescription from a healthcare practitioner;
a consultation offering module to:
first, present, via a graphical user interface, the patient with at least two different consultation visit types suitable for the patient to obtain the prescription for the selected product,
second, receive a selection of a consultation visit type by the patient via the graphical user interface,
third, present, via the graphical user interface, available healthcare practitioner types that offer the patient-selected consultation visit type;
fourth, receive a selection of a type of healthcare practitioner by the patient, and
fifth, present the patient with list of healthcare practitioners enrolled in the system that (i) have authority to provide the prescription, (ii) offer the patient-selected consultation visit type, and (iii) are the patient-selected type of healthcare practitioner;
a selection module to receive, from the patient, a selection of one of the presented healthcare practitioners;
a consultation module to initiate a healthcare consultation of the selected consultation visit type between the patient using the first client device and a healthcare practitioner of the patient-selected type of healthcare practitioner using a second client device;
a prescription generation module to enable the consulting healthcare practitioner to generate a prescription in connection with a completed consultation to enable the patient to purchase at least one of the products that require a prescription; and
a shipping module to facilitate shipping the purchased product to the patient.

2. An inter-practitioner digital health platform, comprising:
a first computing system to:
access a database of a pharmacist provider identifying a plurality of products available at a pharmacy of the pharmacist provider, wherein the plurality of products comprises thousands of products within hundreds of categories of products,
display, via an electronic display, to the pharmacist provider, the plurality of available products via a first graphical user interface rendered by the first computing system; and
display, as part of the first graphical user interface, information characterizing:
a first practice of a first healthcare practitioner, and
a second practice of a second healthcare practitioner;
an input device associated with the first computing system to receive, from the pharmacist provider:
a first curated pharmacist-selection of a first subset of less than all the plurality of available products for selective presentation to the first healthcare practitioner, and
a second curated pharmacist-selection of a second subset of less than all the plurality of available products for selective presentation to the second healthcare practitioner;
an integration subsystem to provide the first healthcare practitioner access to the first curated pharmacist-selection and to provide the second healthcare practitioner access to the second curated pharmacist-selection; and
a unified order requisition portal to:
receive, from the first healthcare practitioner, a first recommended product from the first curated pharmacist-selection for purchase by a first patient;
receive, from the second healthcare practitioner, a second recommended product from the second curated pharmacist-selection for purchase by a second patient;
render, for display on a first patient computing device, the first recommended product as recommended by the first healthcare practitioner alongside a plurality of additional products available at the pharmacy that are identified as being associated with the first recommended product and available for purchase by the first patient; and
render, for display on a second patient computing device, the second recommended product as recommended by the second healthcare practitioner alongside a plurality of additional products available at the pharmacy that are identified as being associated with the second recommended product and available for purchase by the second patient.

3. A system comprising,
a processor; and
a non-transitory computer readable medium with instructions stored thereon that, when executed by the processor, cause a computing device to:
render a first graphical user interface, for display via an electronic display to a pharmacist provider, wherein the first graphical user interface:
displays a plurality of products available for purchase from a pharmacy associated with the pharmacist provider,
identifies a first healthcare provider and a second healthcare provider,
facilitates a curated selection of products by the pharmacist provider of a first subset of available products to be shared with the first healthcare provider, and
facilitates a curated selection by the pharmacist provider of a second subset of available products to be shared with the second healthcare provider;
integrate, via an integration subsystem:
the first curated selection of available products into a first website of the first healthcare provider as an integrated graphical user interface, and
the second curated selection of available products into a second website of the second healthcare provider as an integrated graphical user interface;
receive, via an order requisition portal:
a first recommendation from the first healthcare provider to a first patient that the first patient purchase a first product in the first subset of available products integrated within the graphical user interface of the first website of the first healthcare provider, and
a second recommendation from the second healthcare provider to a second patient that the second patient purchase a second product in the second subset of available products integrated within the graphical user interface of the second website of the second healthcare provider;
receive requests from the first patient and the second patient to purchase the first product and the second product, respectively; and
generate orders within the pharmacy to fulfil the purchases of the first and second products by the first and second patients, respectively.

4. An inter-practitioner digital health platform, comprising:
a pharmacy database of a pharmacist provider that identifies thousands of products within hundreds of categories of products that are available from a pharmacy;
a digital communications network connected to the pharmacy database;
a computing system connected to the communications network to:
access the pharmacy database of the pharmacist provider via the digital communications network,
display, via an electronic display, to the pharmacist provider, the available products via a graphical user interface, and
display, via the electronic display, information identifying a first practice of a first healthcare practitioner and a second practice of a second healthcare practitioner;
an input device associated with the computing system to receive, from the pharmacist provider:
a first curated pharmacist-selection of a first subset of less than all the plurality of available products for selective presentation to the first healthcare practitioner, and
a second curated pharmacist-selection of a second subset of less than all the plurality of available products for selective presentation to the second healthcare practitioner;
an integration subsystem to enable the pharmacist provider to:
share the first curated pharmacist-selected subset of available products with the first healthcare practitioner, and
share the second curated pharmacist-selected subset of available products with the second healthcare practitioner;
an order requisition portal to receive, via the digital communications network:
from the first healthcare practitioner, a recommendation of a first product from the first curated pharmacist-selected subset of healthcare products for purchase by a first patient directly from the pharmacy,
from the second healthcare practitioner, a recommendation of a second product from the second curated pharmacist-selected subset of healthcare products for purchase by the second patient directly from the pharmacy,
from the first patient, a purchase confirmation to purchase the first recommended product, and
from the second patient, a purchase confirmation to purchase the second recommended product; and
a secure communication subsystem to enable the pharmacist provider to communicate securely with the first healthcare practitioner and the first patient regarding the recommended first product and, separately and securely, with the second healthcare practitioner and the second patient regarding the recommended second product.

5. The digital health platform of claim 4, wherein the secure communication subsystem includes a store-and-forward messaging system to enable recorded messages to be securely forwarded between a recipient and a sender.

6. The system of claim 1, wherein the healthcare practitioner comprises one of a medical doctor, a dermatologist, a physician's assistant, and a nurse practitioner.
